(19) United States
(12) Patent Application Publication
(10) Pub. No.: US 2025/0354182 A1
(43) Pub. Date: Nov. 20, 2025

(54) BIOSYNTHESIS OF BIFUNCTIONAL TERPENOIDS

(71) Applicant: The Regents of the University of California, Oakland, CA (US)

(72) Inventors: Junyoung Park, Los Angeles, CA (US); Glenn Nurwono, Los Angeles, CA (US)

(73) Assignee: The Regents of the University of California, Oakland, CA (US)

(21) Appl. No.: 19/199,970

(22) Filed: May 6, 2025

Related U.S. Application Data
(60) Provisional application No. 63/643,017, filed on May 6, 2024.

(51) Int. Cl. C12P 7/44; C12N 9/04; C12N 9/10; C12P 5/00

(52) U.S. Cl. CPC C12P 7/44; C12N 9/0006; C12N 9/1085; C12P 5/007; C12Y 101/01034; C12Y 205/01001

(57) ABSTRACT

A strain of Yarrowia lipolytica was engineered to overexpress β-hydroxy β-methylglutaryl-CoA reductase and farnesyl pyrophosphate synthase, as rate-limiting enzymes in the mevalonate and sesquiterpenoid synthesis pathways respectively. Metabolite extracts from this strain were run on LC-MS and showed a number of novel compounds being produced, including terpenoids varying in lengths and oxidation states. Upon NMR and MS/MS structure validation as well as biochemical assays, these compounds were determined as a new class of non-natural compounds, bifunctional terpenoids. Studies on the overexpression of P450 enzymes, alcohol oxidase, aldehyde dehydrogenase, and alcohol dehydrogenase showed that expression of these enzymes in addition to β-hydroxy β-methylglutaryl-CoA reductase and farnesyl pyrophosphate synthase increase the production of bifunctional terpenoids. Bioactivity assays demonstrate the application of bifunctional terpenoids.

### CROSS REFERENCE TO RELATED APPLICATIONS

This application claims the benefit under 35 U.S.C. Section 119 (e) of co-pending and commonly assigned U.S. Provisional Patent Application No. 63/643,017, filed May 6, 2024, entitled “BIOSYNTHESIS OF TERPENOID DIACIDS”, the contents of which is incorporated by reference herein.

### SEQUENCE LISTING

The instant application contains a Sequence Listing which has been submitted electronically in XML file format and is hereby incorporated by reference in its entirety. Said XML copy, created on Jul. 15, 2025, is named 30435_0478USU1_SL.xml and is 21,310 bytes in size.

### TECHNICAL FIELD

Embodiments of the disclosure concern at least the fields of microbiology and biochemistry.

### BACKGROUND OF THE INVENTION

In yeasts such as Yarrowia lipolytica and many other organisms, farnesyl pyrophosphate (FPP) and geranyl pyrophosphate (GPP) are central precursors to many terpene-derived natural products, including the triterpenoid squalene, and the sterol ergosterol. FPP and GPP are located at a branchpoint in terpenoid metabolism for biosynthesis of important lipids and fungal cell membrane components. Thus, increasing the availability of FPP and GPP would direct carbon fluxes toward non-dominant terpenoid pathways to produce novel molecules.

Over the past several years, yeasts including Y. lipolytica have emerged as increasingly important organisms in biotechnology to produce various commodity chemicals, specialty chemicals, and acetyl-CoA-based natural products such as terpenoids and polyketides. Conventional research often involves overexpression of heterologous biosynthetic enzymes or pathways into a host organism to produce a compound outside of its endogenous metabolism. However, the results of such genetic manipulations are unpredictable, and the terpenoid and polyketide chemical space of microorganisms such as Y. lipolytica has yet to be explored, due in part to the observed high flux towards lipid accumulation.

There is a need in the art for materials and methods useful for the functionalization, production, and use of new terpenoids and the like.

### SUMMARY OF THE INVENTION

As noted above, Yarrowia lipolytica is an oleaginous yeast that is increasingly employed for metabolic engineering and production of various natural products and metabolites, though its propensity for noncanonical terpenoid functionalization is underexplored. As discussed below, we report the discovery of multiple novel bifunctional terpenoid compounds made by increasing the flux through the mevalonate pathway by overexpressing β-hydroxy β-methylglutaryl-CoA reductase (HMGR) and farnesyl pyrophosphate synthase (FPPS). Structural elucidation revealed that the new compounds include oxidized derivatives of farnesol. The additional overexpression of cytochrome P450 enzymes in the CYP52 family increased the production of the various chain length bifunctional terpenoid compounds, derived from geranylgeraniol, geranylfarnesol, and squalene. Interestingly, these P450s (ALK3-7) had some selectivity for different chain length terpenoid compounds.

In illustrative working embodiments of the invention disclosed herein, an engineered strain of Yarrowia lipolytica overexpressing HMGR, FPPS, and ALK5 had a 60-fold increase in the production of a terpenoid diacid over the engineered strain overexpressing only HMGR and FPPS. This finding demonstrates that increased precursor supply and oxidative capacity in microorganisms such as Y. lipolytica unveil the untapped terpenoid chemical space.

The invention disclosed herein has a number of embodiments. Embodiments of the invention include, for example, a composition of matter comprising one or more terpenoid compounds shown in FIG. 8. Optionally such compositions further include a microorganism engineered to overexpress β-hydroxy β-methylglutaryl-CoA reductase (HMGR) and farnesyl pyrophosphate synthase (FPPS). In certain embodiments of the invention, the microorganism is further engineered to overexpress additional proteins such as phosphatases, oxidases, dehydrogenases, P450 enzymes, and the like, for example ALK3, ALK4, ALK5, ALK6 and/or ALK7 for P450 enzymes, Q6C1F6 (YALI0F16709g) for phosphatase, Q6CCQ8 (YALI0C07414g) for alcohol dehydrogenase, and Q6CG32 (YALI0B01298g) for aldehyde dehydrogenase. In some embodiments of the invention, the terpenoid diacid comprises an isoprenoic diacid, a geranoic diacid, a geranylgeranoic diacid, a geranylfarnesoic diacid and a squalene diacid. In certain embodiments of the invention, the terpenoid diacid comprises a compound having the general structures:

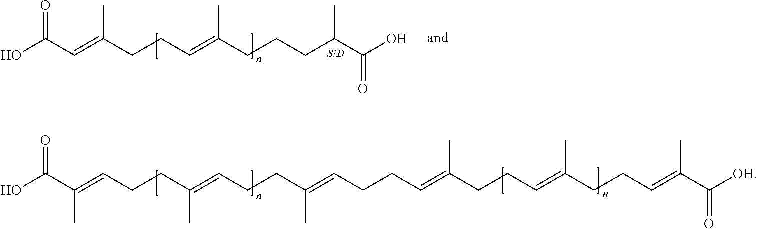

In some embodiments of the invention, the bifunctional terpenoid comprises a terpene having various oxidation states of its termini with the general structures:

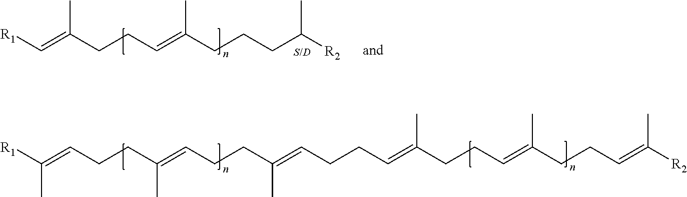

where R₁ and R₂ are each CH₃, CH₂OH, CHO, or CO₂H, and n is a whole number.

Embodiments of the invention also include compositions of matter comprising a microorganism making a terpenoid diacid, wherein when disposed in YPD and YNB culture media at 30° C., the microorganism can make the bifunctional terpenoid such that its concentration is at least 0.1, 0.5, 1 or 10 milligrams/L, distributed in the microbial cells as well as the culture media.

Embodiments of the invention can utilize a variety of different microorganisms (see, e.g., Patel et al., Microorganisms. 2020 Mar. 19; 8 (3): 434). In typical embodiments of the invention, the microorganism is a yeast. Optionally, for example, the microorganism is a Yarrowia, Saccharomyces, Candida, Rhodosporidium, Cryptococcus, Rhodotorula, Lipomyces, or Trichosporon yeast species. In such embodiments of the invention, the microorganism comprises exogenous/altered nucleic acid sequences that increase the expression of β-hydroxy β-methylglutaryl-CoA reductase and farnesyl pyrophosphate synthase in the microorganism. In certain embodiments of the invention, the microorganism is a bacteria such as Escherichia, Methylobacterium, or Rhodococcus. In certain embodiments of the invention, the microorganism also comprises exogenous/altered nucleic acid sequences that increase the expression of additional proteins such as phosphatases, oxidases, dehydrogenases, and P450 enzymes and the like, for example ALK3, ALK3, ALK4, ALK5, ALK6, ALK7, Q6C1F6 (YALI0F16709g), Q6CCQ8 (YALI0C07414g), and/or Q6CG32 (YALI0B01298g) polypeptides in the microorganism.

Embodiments of the invention also include methods of making a bifunctional terpenoid using the engineered microorganisms disclosed herein. In illustrative embodiments, these methods comprise combining a microorganism with a culture media, wherein the microorganism is selected to comprise exogenous/altered nucleic acid sequences selected to increase the expression of β-hydroxy β-methylglutaryl-CoA reductase and farnesyl pyrophosphate synthase polypeptides in the microorganism; and the culture media is selected to allow the production of the terpenoid diacid when the microorganism is disposed therein; such that the bifunctional terpenoid is made. In typical embodiments of the invention, the microorganism is further selected to comprise exogenous/altered nucleic acid sequences selected to increase the expression of ALK3, ALK4, ALK5, ALK6, ALK7, Q6C1F6 (YALI0F16709g), Q6CCQ8 (YALI0C07414g), and/or Q6CG32 (YALI0B01298g) polypeptides in the engineered microorganism. Optionally, the microorganism is a Yarrowia, Saccharomyces, Candida, Rhodosporidium, Cryptococcus, Rhodotorula, Lipomyces, or Trichosporon yeast species. In addition, a wide variety of different medias can be used, depending upon the microorganism selected (see, e.g., Yeast: Molecular and Cell Biology 2nd Edition by Horst Feldmann (Editor); and Yeast Biotechnology by G. C. Stewart). In certain embodiments of the invention, the culture media comprises a yeast peptone dextrose (YPD) culture media or a yeast nitrogen base (YNB) culture media. In certain embodiments of the invention, the media comprises precursor molecules geraniol and farnesol. Optionally in such methods, amounts of the bifunctional terpenoid made by the microorganisms growing in the culture media are at least 0.1, 0.5, 1 or 10 milligrams/L. Certain embodiments of these methods include the steps of purifying the bifunctional terpenoids and/or performing additional chemical modifications to the bifunctional terpenoids made by the microorganism. For example, certain embodiments of these methods include performing a purification process on the bifunctional terpenoid made by the microorganism. Other embodiments of the invention can further include performing a polymerization process on the bifunctional terpenoids. Other embodiments of the invention can further include making a derivative of a bifunctional terpenoid made by the microorganism.

Embodiments of the invention also include methods of using a bifunctional terpenoid disclosed herein, for example to increase microbial cell growth and/or inhibit mammalian cell growth as shown in FIG. 7.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: Discovery of a new terpenoid-derived diacid. FIG. 1(a): Culture extracts at different timepoints were taken, showing the fold change of the engineered strain compared to the wild-type strain (3 biological replicates). Overexpressed enzymes in the engineered strain are highlighted in bold. FIG. 1(b): Peak lists of the culture extract of the engineered strain and the wild-type strain were compared using KD-Tree for nearest-neighbor (RT tolerance: 0.2 min, m z tolerance: 0.002). Data for unique peaks found in the engineered strain are shown, highlighting the top 4 hits. FIG. 1(c): Extracted ion chromatogram with traces from the wild-type culture extract and the engineered strain extract, showing the identified new peak and the resulting NMR-verified structure.

FIG. 2: Discovery of related biosynthetic intermediates. FIG. 2(a): Biosynthetic logic of farnesoic diacid follows oxidation at both termini. Oxidation happens sequentially at either terminus. The backbones include the 10,11-ene species (denoted with a) and the 10,11-dihydro species (denoted with b). FIG. 2(b): The extracted ion chromatograms (EICs) from engineered culture extract corresponding to the m z of prospective biosynthetic intermediates. Retention times are denoted for compounds that had structural verification from NMR, MS/MS, or a standard. FIG. 2(c): Respective observed biosynthetic intermediates in culture extracts and their structural determination method.

FIG. 3: Individual biochemical characterization of biosynthetic enzymes identified. FIG. 3(a): S. cerevisiae overexpressing ALK5 was fed 1a, resulting in biotransformation to 6a. EIC of experimental and control reactions are shown for the product, 6a. FIG. 3(b): Purified alcohol oxidase (AOX) was used in an in vitro reaction with 2a, resulting in production of previously undetected 9a. EIC of experimental and control reactions are shown for the substrate (6a) and the product (9a). FIG. 3(c): E. coli overexpressing aldehyde dehydrogenase (ALDH2) was lysed and fed 9a, resulting in transformation to 12a. EIC of experimental and control reactions are shown for the substrate (9a) and the product (12a). FIG. 3(d): Overexpression of biosynthetic enzymes in Y. lipolytica results in increased production of the final compound 12a.

FIG. 4: Reconstitution of 12a/b biosynthetic pathway and substrate preference of biosynthetic enzymes. FIG. 4(a): Biosynthetic enzymes were expressed stepwise in S. cerevisiae. EIC traces corresponding to the m/z of confirmed biosynthetic intermediates are shown. FIG. 4(b): S. cerevisiae overexpressing ALK5, AOX, or both enzymes were fed farnesol (3 biological replicates). Resulting ion counts of 3a and 6a are shown for several timepoints. FIG. 4(c): A biosynthetic pathway from FPP to 12a is determined with enzymes and cofactors. ALK5 is sufficient to produce 6a, but AOX can also produce 3a, which can be a substrate for ALK5 to produce 6a. FIG. 4(d): A bidirectional biosynthetic network from 1a/b to 12a/b is shown with confirmed and proposed enzymes.

FIG. 5: Biosynthetic logic and intermediates for longer chain terpenoid diacids. FIG. 5(a): Longer terpenoid precursors are built upon FPP, leading to other new compounds. Biosynthetic pathways of C20 FIG. 5(b), C25 FIG. 5(c), and C30 FIG. 5(d) terpenoid diacids. Intermediates follow similar logic with oxidation at both termini, with or without the reduction of the double bond.

FIG. 6: Longer terpenoid diacids produced by ALK6. FIG. 6(a): LC-MS analysis of culture extract using a strain over-expressing HMGR+FPPS+ALK5. EIC traces corresponding to the m z of prospective biosynthetic intermediates are shown. Retention times are denoted for compounds that had structural verification from NMR or MS/MS. FIG. 6(b): Respective observed biosynthetic intermediates in culture extracts and their structural determination method. FIG. 6(c): Production of NMR-verified longer terpenoid diacids between strains varying in expression of different ALK proteins are shown. Statistical significance from multiple-comparison one-way ANOVA and Turkey's HSD is shown.

FIG. 7: Bioactivity of farnesoic diacid. FIG. 7(a): E. coli NCM372 was grown to an optical density (OD₆₀₀) of around 0.2, where either 12a in DMSO was added to final concentration of 10 μM (+) or DMSO was added (−). Three biological replicates are shown, and statistical significance using an unpaired t test is shown. FIG. 7(b): Immortalized baby mouse kidney (iBMK) cells were grown, either non-activated (D3) or Ras-activated (Ras). 12a in DMSO was added to final concentration of 10 μM (+) or DMSO was added (−). Three biological replicates are shown, and statistical significance using an unpaired t test is shown. These studies show that microbial cells grew ˜10% faster with the terpenoid diacid treatment, while cancer-like cell lines grew ˜10% slower with the terpenoid diacid treatment.

FIG. 8: Diagrams of chemical structures of the compounds of the invention.

FIG. 9: Schematics showing illustrative derivatizations of terpenoid diacids using farnesoic diacid as an example.

FIG. 10: Schematics showing illustrative derivatizations hydroxyacid intermediates using 12-hydroxyfarnesoic acid as an example.

FIG. 11: Structures of proposed C20, C25, and C30 diacid derivatives containing a carbocycle. Structures resulting from cyclization at the ω-termini and α-termini.

FIGS. 12-49: Nuclear magnetic resonance spectroscopy of novel bifunctional terpenoids of the invention.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Metabolism encompasses a vast and valuable repository of chemicals and reactions within biological organisms. Primary metabolism, involving reactions essential for growth and life, is well understood and largely conserved across all kingdoms of life. In contrast, secondary metabolism, which comprises specialized reactions for environmental interactions, remains underexplored. The molecules produced by secondary metabolism, commonly known as natural products (NPs), form a diverse chemical space that provides ecological advantages to the producing organisms. Some of these NPs include pheromones for chemical communication, siderophores for iron sequestration, and defense molecules to deter herbivores.

NPs encompass a wide array of bioactive molecules valuable to the pharmaceutical, agricultural, and nutrition industries. One of the most important discoveries of the 20th century is the antibiotic penicillin, produced by Penicillium, which has served as a life-saving drug for humans. Similarly, the antiparasitic drug ivermectin, derived from various soil bacteria, provides significant therapeutic benefits to humans with parasitic-borne diseases. These biochemical landmarks are attributed to serendipity. Thus, developing systematic strategies to navigate the extensive chemical space of secondary metabolism is crucial for uncovering new NPs as well as synthesizing new non-natural products (NNPs).

NPs and NNPs stem from a select few building blocks that are coupled to form general carbon scaffolds. Polyketides are derived from acyl-CoA units, terpenoids are built from isoprenyl units, and amino acids afford non-ribosomal peptides and phenylpropanoids. These general carbon backbones are then further functionalized by biosynthetic enzymes often specific to the organism, yielding a vast diversity of NPs with much more diverse functions and bioactivity than their core structures. Furthermore, several important NPs are biosynthesized through convergent pathways, featuring building blocks from different classes. These additional features, such as glycosyl groups for recognition and isoprenyl moieties for altered solubility, enhance their functionality both for the producer and for human applications. Thus, from a few building blocks, there is a wide variety of NPs and NNPs through shared precursors and distinct biosynthetic enzymes.

Building upon the idea of a limited yet shared pool of precursors potentially limiting (N)NP production, we sought to increase biosynthetic precursor availability to directly induce metabolic conditions favorable for (N)NP production and discovery. Instead of relying on modifying culture conditions to trigger production, genetic engineering can allow for the rational manipulation of specific enzymes to alter metabolic flux, such as overexpressing enzymes that are rate-limiting in biosynthesis. Using mass spectrometry-based detection, the metabolomes of modified and unmodified strains can be compared, enabling the identification of candidate compounds with much greater sensitivity than traditional bioactivity assays. This approach can be particularly fruitful for genetically tractable organisms by investigating their carbon yield, or the carbon output as the target product per input substrate carbon. With advancements in genetic tools, the large-scale production of (N)NPs through metabolic engineering has become accessible, yet carbon yield remains low as not all carbons are accounted for. By tracking biosynthetic precursor utilization with untargeted metabolomics, this is an orthogonal method to discover (N)NPs only accessible through a genetic manipulation of carbon flux.

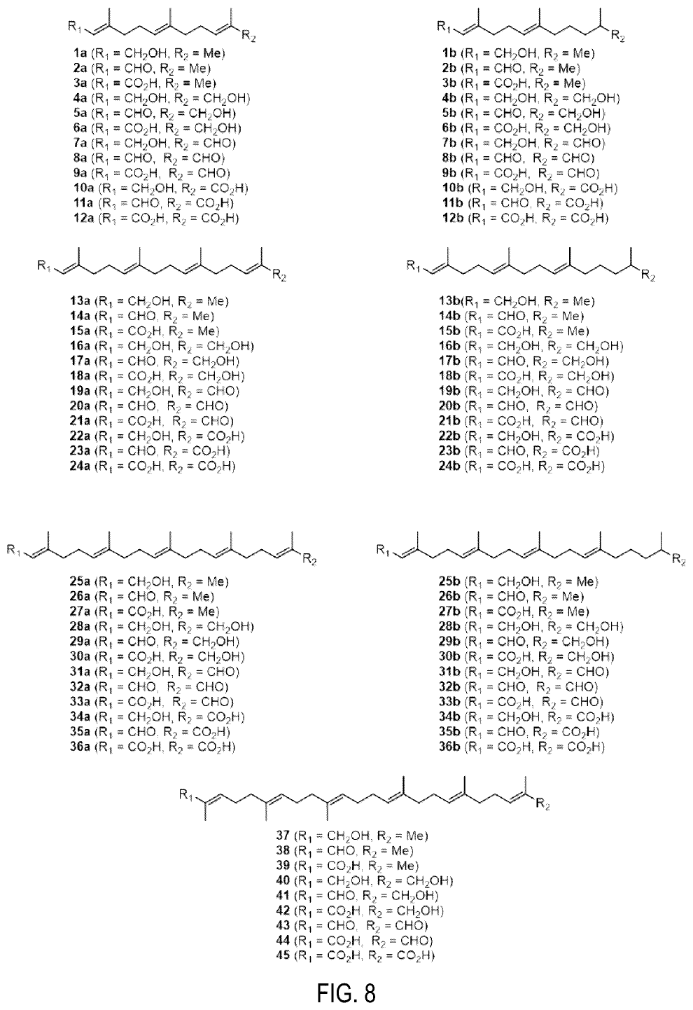

Leveraging this carbon flux-pushing metabolome mining strategy, we performed untargeted metabolomics on a metabolically engineered chemical space and discovered terpenoids that, to our knowledge, have not been reported before (see, e.g. FIG. 8). By enhancing terpenoid precursor availability through the overexpression of key rate-limiting enzymes in the mevalonate pathway and sesquiterpenoid synthesis, we identified several novel terpenoid compounds, demonstrating the impact of increased precursor availability. Furthermore, overexpression of key biosynthetic enzymes facilitates production of a wide pool of NNPs consisting of bifunctional compounds varying in oxidation state and lengths. These findings reveal that the expanded precursor pool facilitates biosynthesis of new NNPs. Our work highlights the significance and potential of employing genetic engineering and metabolomics to probe (N)NP production. Our discoveries of new bifunctional terpenoids and the metabolic adaptability of Y. lipolytica through its enzymes reveal a novel pool of NNPs that can be accessed by modifying precursor flux and availability.

Yarrowia lipolytica is often employed for heterologous expression and production of various lipids and metabolites. However, its terpenoid metabolism is underexplored. Here, we report the production of novel terpenoid compounds being produced through overexpression of β-Hydroxy β-methylglutaryl-CoA reductase (HMGR) and farnesyl diphosphate synthase (FPPS). Structure elucidation revealed that the new compound was a diacid derivative of farnesol, and overexpression of cytochrome P450 enzymes in the CYP52 family increased production of the compound and various other diacids. Interestingly, these P450s (ALK3-7) had different selectivity for different chain length bifunctional terpenoids. Our final engineered strain overexpressing HMGR, FPPS, and ALK5 had a 60-fold increase over the strain overexpressing only HMGR and FPPS. Our findings demonstrate that increased precursor supply and oxidative capacity in microorganisms such as Y. lipolytica unveil the untapped terpenoid chemical space.

Over the past several years, Yarrowia lipolytica has emerged as a prominent industrial organism in biotechnology to produce various commodity chemicals and acetyl-CoA-based terpenoids and polyketides. This research often involves overexpression of heterologous biosynthetic enzymes or pathways to produce a compound outside of its endogenous metabolism. However, the terpenoid and polyketide chemical space has yet to be fully mapped.

In Y. lipolytica and many other organisms, farnesyl diphosphate (FPP) is a central precursor to many terpene-derived natural products, including the triterpenoid squalene, and the sterol ergosterol. Its role in the biosynthesis of important lipids and fungal cell membranes components makes FPP a branchpoint in terpenoid metabolism, with little accumulation in the Y. lipolytica. Thus, an increase of FPP availability may allow for direction of flux towards unmapped pathways to produce novel molecules.

We set out to examine whether Y. lipolytica is capable of producing any novel terpenoids through an increased precursor FPP supply. In our investigation, we discovered and isolated a number of compounds including a new compound termed farnesoic diacid, a derivative of farnesol with carboxylic acid functionality at both termini of the molecule. We identified several cytochrome P450 enzymes (ALK3-7) that are involved in the biosynthesis of farnesoic diacid. The novel compounds were further confirmed by analyzing tandem mass spectrometry (MS/MS) fragmentation patterns and NMR. By combining the engineering strategies of increased precursor supply and increased expression of relevant biosynthetic enzymes, we designed a strain to overexpress HMGR, FPPS, and ALK5 resulting in a substantial accumulation of farnesoic diacid. To our knowledge, this is the first observed biosynthesis of farnesoic diacid in Y. lipolytica, representing the viability in activating silent biosynthetic pathways through increasing precursor availability.

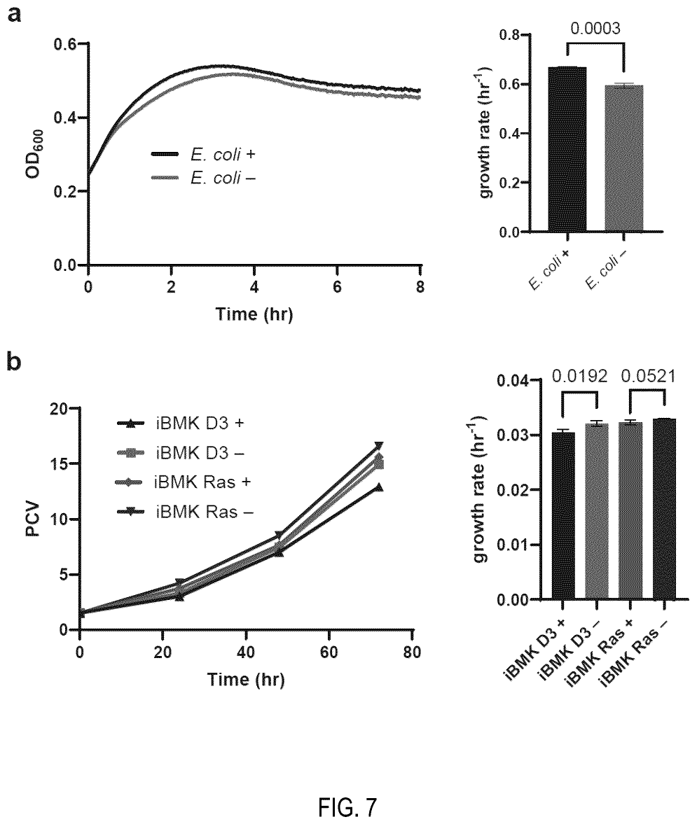

As shown in FIG. 8, we discovered a number of bifunctional terpenoids that are synthesized by engineered Yarrowia lipolytica cells. We experimentally verified their existence and their structure by liquid chromatography-mass spectrometry (LC-MS), nuclear magnetic resonance spectroscopy (NMR), high-resolution MS/MS, and biochemical assays. These molecules are of terpenoid origin with various functionalities at a and @ termini. We reconstituted their biosynthetic routes and fully mapped the novel bifunctional terpenoid pathway. As shown in FIG. 7, we discovered that one of our novel molecules, farnesoic diacid, increases microbial cell growth and decreases mammalian (cancer-like) cell growth. These findings provide evidence for the potential application of our novel molecules in the biotechnology industry to enhance the growth of microorganisms for bioproduct synthesis and to inhibit cancer growth. Our novel molecules also share structural similarity with oxylipin and polyunsaturated fatty acids, which have been implicated in immunity and obesity in humans, and juvenile hormones, which are implicated in insect development. Furthermore, with the varying lengths of carbon backbones and functionalization on both termini, our novel molecules are poised to bring about new materials as well as new bioactivities.

The invention disclosed herein has a number of embodiments. Embodiments of the invention include, for example, a composition of matter comprising one or more compounds shown in FIG. 8. Optionally the compositions can further comprise one or more pharmaceutically acceptable excipients such as a buffering agent and/or an antimicrobial agent. Such pharmaceutically acceptable excipients are well known in that art and a thorough discussion of pharmaceutically acceptable carriers, diluents, and other excipients is presented in Remington's Pharmaceutical Sciences (Mack Pub. Co., N.J. current edition).

Embodiments of the invention include a microorganism engineered to make a bifunctional terpenoid compound. Typically such compositions include a microorganism engineered to overexpress β-hydroxy β-methylglutaryl-CoA reductase (HMGR) and farnesyl pyrophosphate synthase (FPPS). In certain embodiments of the invention, the microorganism is further engineered to overexpress additional proteins such as phosphatases, oxidases, dehydrogenases, P450 enzymes, and the like, for example ALK3, ALK4, ALK5, ALK6 and/or ALK7 for P450 enzymes, Q6C1F6 (YALI0F16709g) for phosphatase, Q6CCQ8 (YALI0C07414g) for alcohol dehydrogenase, and Q6CG32 (YALI0B01298g) for aldehyde dehydrogenase. In some embodiments of the invention, the compound comprises an isoprenoic diacid, a geranoic diacid, geranoic diacid, a geranylgeranoic diacid, a geranylfarnesoic diacid and a squalene diacid. In certain embodiments of the invention, the terpenoid diacid comprises a compound having the general structures where n is a whole number:

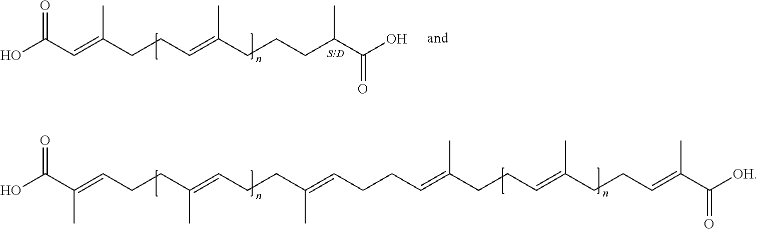

In some embodiments of the invention, the bifunctional terpenoid comprises a terpene having various oxidation states of its termini with the general structures and formulas:

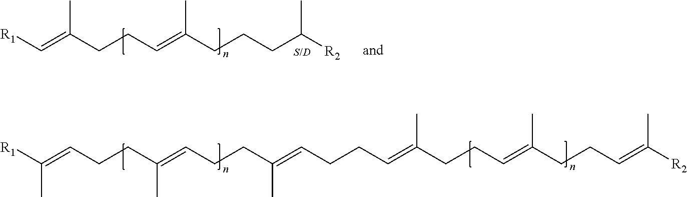

where R₁ and R₂ are each CH₃, CH₂OH, CHO, or CO₂H, and n is a whole number. Compounds made by the methods of the invention include those shown in Table 1 below.

TABLE 1
COMPOUNDS OF THE INVENTION

| Formula | R | Dihydro (Y/N) |
|---|---|---|
| C₅ₙH(₈ₙ₋₂)O₄ | R₁ = R₂ = CO₂H | N |
| C₅ₙH₈ₙO₄ | R₁ = R₂ = CO₂H | Y |
| C₅ₙH(₈ₙ₊₂)O | R₁ = CH₂OH, R₂ = Me | N |
| C₅ₙH(₈ₙ₊₄)O | R₁ = CH₂OH, R₂ = Me | Y |
| C₅ₙH₈ₙO | R₁ = CHO, R₂ = Me | N |
| C₅ₙH(₈ₙ₊₂)O | R₁ = CHO, R₂ = Me | Y |
| C₅ₙH₈ₙO₂ | R₁ = CO₂H, R₂ = Me | N |
| C₅ₙH(₈ₙ₊₂)O₂ | R₁ = CO₂H, R₂ = Me | Y |
| C₅ₙH(₈ₙ₊₂)O₂ | R₁ = CH₂OH, R₂ = CH₂OH | N |
| C₅ₙH(₈ₙ₊₄)O₂ | R₁ = CH₂OH, R₂ = CH₂OH | Y |
| C₅ₙH₈ₙO₂ | R₁ = CHO, R₂ = CH₂OH or | N |
| | R₁ = CH₂OH, R₂ = CHO | |
| C₅ₙH(₈ₙ₊₂)O₂ | R₁ = CHO, R₂ = CH₂OH or | Y |
| | R₁ = CH₂OH, R₂ = CHO | |
| C₅ₙH₈ₙO₃ | R₁ = CO₂H, R₂ = CH₂OH or | N |
| | R₁ = CH₂OH, R₂ = CO₂H | |
| C₅ₙH(₈ₙ₊₂)O₃ | R₁ = CO₂H, R₂ = CH₂OH or | Y |
| | R₁ = CH₂OH, R₂ = CO₂H | |
| C₅ₙH(₈ₙ₋₂)O₂ | R₁ = R₂ = CHO | N |
| C₅ₙH₈ₙO₂ | R₁ = R₂ = CHO | Y |
| C₅ₙH(₈ₙ₋₂)O₃ | R₁ = CO₂H, R₂ = CHO or | N |
| | R₁ = CHO, R₂ = CO₂H | |
| C₅ₙH₈ₙO₃ | R₁ = CO₂H, R₂ = CHO or | Y |
| | R₁ = CHO, R₂ = CO₂H | |

Embodiments of the invention also include compositions of matter comprising a microorganism making a bifunctional terpenoid, wherein when disposed in YPD and YNB culture media at 30° C., the microorganism can make the bifunctional terpenoid such that concentration of the bifunctional terpenoid is at least 0.1, 0.5, 1 or 10 milligrams/L, distributed in the microbial cells as well as the culture media.

Embodiments of the invention can utilize a variety of different microorganisms (see, e.g., Patel et al., Microorganisms. 2020 Mar. 19; 8 (3): 434). In typical embodiments of the invention, the microorganism is a yeast. Optionally, for example, the microorganism is a Yarrowia, Saccharomyces, Candida, Rhodosporidium, Cryptococcus, Rhodotorula, Lipomyces, or Trichosporon yeast species. In such embodiments of the invention, the microorganism comprises exogenous/altered nucleic acid sequences that increase the expression of β-hydroxy β-methylglutaryl-CoA reductase and farnesyl pyrophosphate synthase in the microorganism. In certain embodiments of the invention, the microorganism is a bacteria such as Escherichia, Methylobacterium, or Rhodococcus. In certain embodiments of the invention, the microorganism also comprises exogenous/altered nucleic acid sequences that increase the expression of additional proteins such as phosphatases, oxidases, dehydrogenases, and P450 enzymes and the like, for example ALK3, ALK3, ALK4, ALK5, ALK6, ALK7, Q6C1F6 (YALI0F16709g), Q6CCQ8 (YALI0C07414g), and/or Q6CG32 (YALI0B01298g) polypeptides in the microorganism.

Embodiments of the invention also include methods of making a bifunctional terpenoid disclosed herein (see, e.g. FIG. 8) using the engineered microorganisms disclosed herein. In illustrative embodiments, these methods comprise combining a microorganism with a culture media, wherein the microorganism is selected to comprise exogenous/altered nucleic acid sequences selected to increase the expression of β-hydroxy β-methylglutaryl-CoA reductase and farnesyl pyrophosphate synthase polypeptides in the microorganism; and the culture media is selected to allow the production of the bifunctional terpenoid when the microorganism is disposed therein; such that the bifunctional terpenoid is made. In typical embodiments of the invention, the microorganism is further selected to comprise exogenous/altered nucleic acid sequences selected to increase the expression of ALK3, ALK4, ALK5, ALK6, ALK7, Q6C1F6 (YALI0F16709g), Q6CCQ8 (YALI0C07414g), and/or Q6CG32 (YALI0B01298g) polypeptides in the engineered microorganism. Optionally, the microorganism is a Yarrowia, Saccharomyces, Candida, Rhodosporidium, Cryptococcus, Rhodotorula, Lipomyces, or Trichosporon yeast species. In addition, a wide variety of different medias can be used, depending upon the microorganism selected (see, e.g., Yeast: Molecular and Cell Biology 2nd Edition by Horst Feldmann (Editor); and Yeast Biotechnology by G. C. Stewart). In certain embodiments of the invention, the culture media comprises a yeast peptone dextrose (YPD) culture media or a yeast nitrogen base (YNB) culture media. In certain embodiments of the invention, the media comprises precursor molecules geraniol and farnesol. Optionally in such methods, amounts of the bifunctional terpenoid made by the microorganisms growing in the culture media are at least 0.1, 0.5, 1 or 10 milligrams/L. Certain embodiments of these methods include the steps of purifying the bifunctional terpenoids and/or performing additional chemical modifications to the bifunctional terpenoids made by the microorganism. For example, certain embodiments of these methods include performing a purification process on the bifunctional terpenoid made by the microorganism. Other embodiments of the invention can further include performing a polymerization process on the bifunctional terpenoids. Other embodiments of the invention can further include making a derivative of a bifunctional terpenoid made by the microorganism. Further embodiments of the invention include methods to overexpress biosynthetic enzymes in Saccharomyces, Escherichia, or other heterologous hosts for enzyme overexpression and purification for biochemical assays and the in vitro production of bifunctional terpenoids (e.g., biotransformation/feeding or in vitro enzymatic production).

Embodiments of the invention include methods of using a bifunctional terpenoid disclosed herein, for example to increase microbial cell growth and/or inhibit mammalian cell growth as shown in FIG. 7. Such methods comprise disposing a bifunctional terpenoid disclosed herein into a microbial culture and/or a mammalian cell culture such that microbial cell growth is increased and/or mammalian kidney cell growth is inhibited. As shown in FIG. 7, treating E. coli with farnesoic diacid increased microbial growth rate and the total biomass content. E. coli is one of the most widely used organisms in industrial biotechnology because of their rapid growth and fast metabolism. Therefore, total addressable market (TAM) is immense. E. coli is used to produce >30% of all FDA-approved recombinant pharmaceuticals. For example, farnesoic diacid has the potential to lower the fermenter operating expenses by 10%. Other microorganisms including yeast may also respond similarly to farnesoic diacid treatment, which would expand TAM.

In addition, the molecules that we discovered represent an exciting pool of organic building blocks that for novel polymer synthesis with desaturated branched-chains, variable chain lengths, and terminal functionalities. Furthermore, the molecules that we discovered may be used as effectors of mammalian cell lipid metabolism and signaling molecules in methods of treating cancer-like mammalian cells with farnesoic diacid to decrease cell growth rate.

As discussed in detail below, in illustrative working embodiments of the invention, a strain of Yarrowia lipolytica PO1f (ATCC MYA-2613) was engineered to overexpress β-hydroxy β-methylglutaryl-CoA reductase (YALI0E04807g) and farnesyl pyrophosphate (FPP) synthase (YALI0E05753g), as rate-limiting enzymes in the mevalonate pathway and sesquiterpenoid synthesis respectively. Metabolite extractions from this strain were run on liquid chromatography-mass spectrometry (LC-MS) and showed a novel compound being produced, with a molecular weight of about 266 g/mol. To elucidate the structure, eight liters of the strain was cultured and about 4 mg of this compound was purified. Upon NMR structure validation, this compound was termed farnesoic diacid, as it resembles farnesol with two terminal carboxylic acids. Currently, its biosynthetic enzymes are known, with one of potential pathway known. Several P450 enzymes were screened. These include ALK3 (YALI0E23474p), ALK4 (YALI0B13816p), ALK5 (YALI0B13838p), ALK6 (YALI0B01848p), ALK5 (YALI0A15488p), which are P450 enzymes that are involved in the conversion of dodecanoic acid to dodecanedioc acid due to structural similarity of proposed substrates. The biosynthesis follows α-oxidation via ALK5 and AOX to farnesoic acid, followed by ω-hydroxylation by ALK5 to 12-hydroxyfarnesoic acid. Then AOX and ALDH2 will oxidize the ω-termini to the aldehyde and acid, respectively. Other alcohol dehydrogenases may also be involved in alcohol oxidation at either terminus, representing another branch in the biosynthetic pathway. Compounds derived from squalene, geranylfarnesol, geranylgeraniol, geraniol, and isoprenol were also seen from metabolite extractions, and several were identified and confirmed via NMR, providing evidence of wide substrate usage when it comes to carbon chain lengths.

Briefly, our studies initially set out to examine whether Y. lipolytica is capable of producing any novel terpenoids through an increased precursor FPP supply. In our investigation, we discovered and isolated a new compound termed farnesoic diacid, a derivative of farnesol with carboxylic acid functionality at both ends of the molecule. We identified several cytochrome P450 enzymes (ALK3-7) that are implicated in the biosynthesis of farnesoic diacid. By combining the engineering strategies of increased precursor pools and increased expression of relevant biosynthetic enzymes, we designed a strain to overexpress HMGR, FPPS, and ALK5 resulting in a substantial farnesoic diacid production. We mapped its biosynthetic pathway by characterizing each enzyme individually, as well as conferring the full pathway in a heterologous host for de novo production of farnesoic diacid. We also identified a number of peaks corresponding to novel bifunctional terpenoids through overexpression of key biosynthetic enzymes, demonstrating a new class of NNPs being produced as a result of rational strain engineering. To our knowledge, this is the first observed biosynthesis of farnesoic diacid, other terpenoid diacids, and bifunctional terpenoids in Y. lipolytica and other microorganisms. Our work constitutes a general metabolic engineering strategy for discovering novel biosynthetic pathways through increasing precursor supply and oxidative capacity in microorganisms.

### EXAMPLES

### Example 1: Biosynthesis of Novel Bifunctional Terpenoids in Yarrowia Lipolytica

The metabolome encompasses a diverse array of small molecules with bioenergetic, biosynthetic, and specialized functions. Secondary metabolism produces numerous specialized metabolites; however, we have yet to complete the map of its biochemical networks and realize its full potential. Here, we map the uncharted terpenoid chemical space using an engineered oleaginous yeast Yarrowia lipolytica. Pushing carbon flux through the mevalonate pathway by overexpression of its rate-determining steps results in various functionalities on terpene backbones. Using nuclear magnetic resonance spectroscopy, mass spectrometry, and biochemical assays, we uncover a novel class of terpenoids that are variously functionalized at both termini. We reconstitute their biosynthetic routes and provide a glimpse of their bioactivities in bacteria and cancer cells. We discover that one of our novel molecules, farnesoic diacid, increases microbial cell growth and decreases mammalian (cancer-like) cell growth. These findings suggest potential application of our novel molecules in the biotechnology industry to enhance the growth of microorganisms for bioproduct synthesis and to inhibit cancer growth. Other bifunctional terpenoids present a growing arsenal of biochemical compounds with novel bioactivities.

### Enhanced Terpenoid Precursor Flux Yields a Novel Farnesol-Derived Diacid

A key characteristic of Y. lipolytica is its inherent hydrophobic metabolism and intracellular environment, which enables the production of lipid droplets and ample supply of acetyl-CoA. To investigate and engineer this hydrophobic chemical space, we overexpressed 3-hydroxy-3-methyl-glutaryl-coenzyme A reductase (HMGR) and farnesyl pyrophosphate synthase (FPPS), which are rate-limiting enzymes in the mevalonate pathway and sesquiterpenoid synthesis¹ respectively.

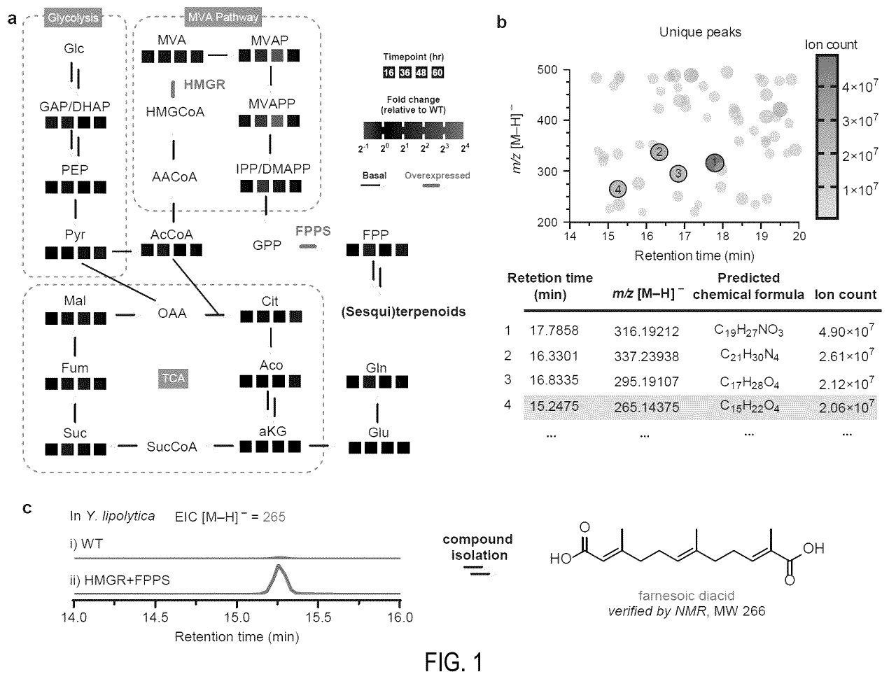

We began by profiling the metabolome of our engineered strain of Y. lipolytica and comparing it to the wild-type at two points during both the exponential and lipogenic phases (FIG. 1a). At the second timepoint, we observed a 1.7-fold increase in acetyl-CoA accumulation, a crucial intermediate for the biosynthesis of lipids, terpenoids, and other hydrophobic compounds. Subsequent timepoints showed lower fold increases in acetyl-CoA, suggesting its utilization in downstream pathways. Further downstream of central carbon metabolism, we observed more pronounced changes in intermediates of the mevalonate pathway and terpenoid synthesis. Specifically, mevalonate, the direct product of HMGR, and its phosphorylated derivatives exhibited increased accumulation compared to the wild-type during the second and third timepoints, with MVAPP showing up to an 11-fold increase. Similarly, FPP accumulated at higher levels, up to a 2-fold increase during the second timepoint. However, by the fourth timepoint, the levels of these terpenoid precursors in the engineered strain were comparable to or lower than those in the wild-type, suggesting the activation of secondary metabolic pathways during the lipogenic phase that consume these precursors. This data indicates that our engineered strain exhibits enhanced metabolic activity through the mevalonate pathway and terpenoid metabolism, with accumulated precursors being consumed as the lipogenic phase progresses.

With confirmation that our engineered strain exhibited increased mevalonate and terpenoid flux, we aimed to identify novel compounds within the engineered strain's chemical space. LC-MS peaks from both the wild-type and engineered strains were collected from extractions from whole cultures three days post-inoculation, revealing unique peaks in the engineered strain. Filtering for plausible terpenoid monoisotopic mass-to-charge ratios (m z) and retention times identified four prominent unique peaks (FIG. 1b). We assigned predicted molecular formulas that were supported by both the compound m z and ¹³C isotope natural abundance. Among these, we focused on the peak corresponding to the molecular formula C₁₅H₂₂O₄, as the most likely compound to be derived from FPP due to both having 15 carbons.

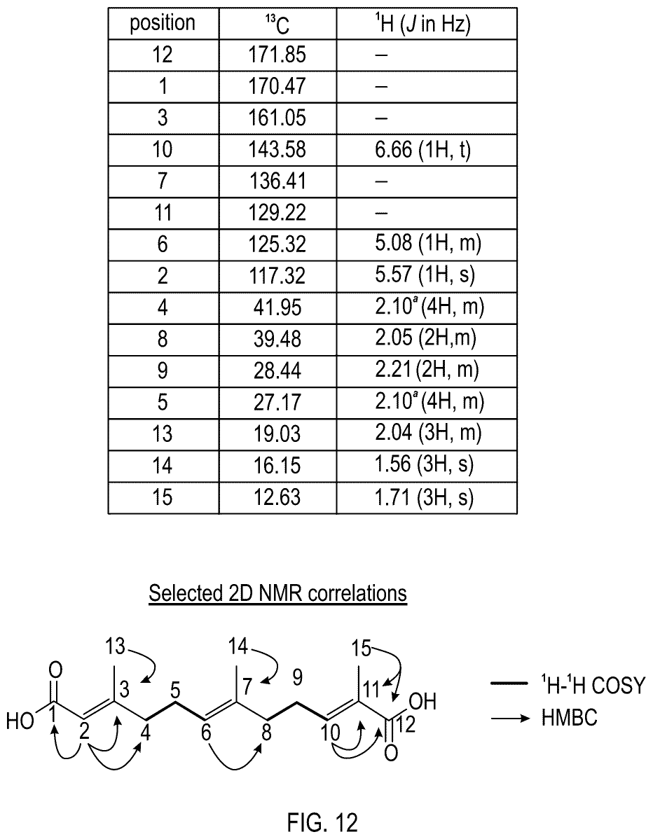

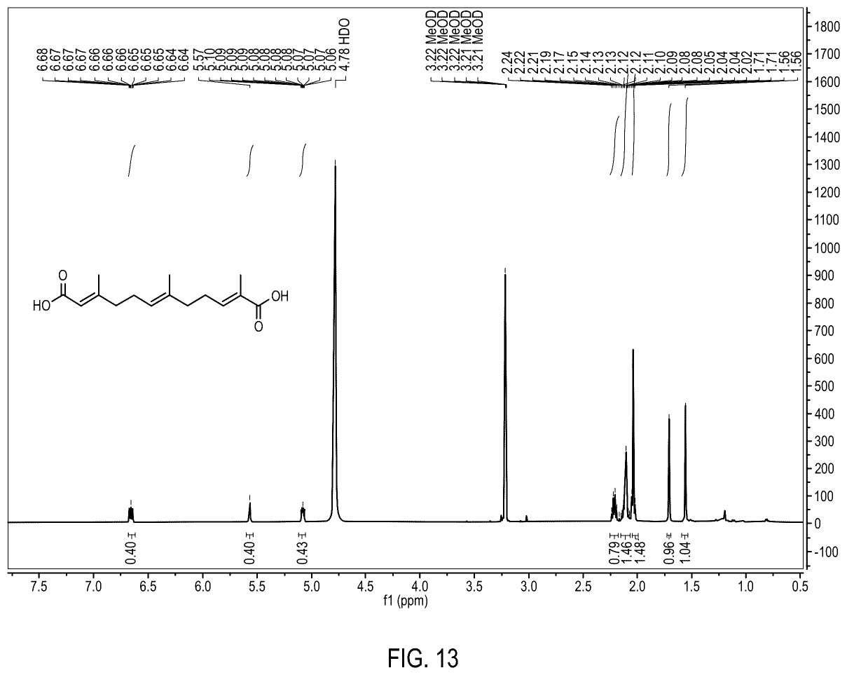

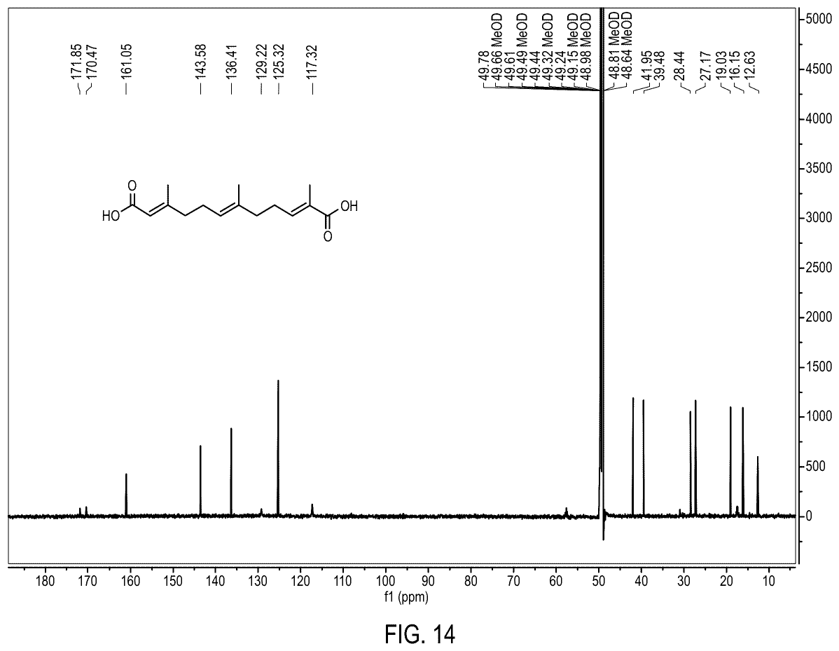

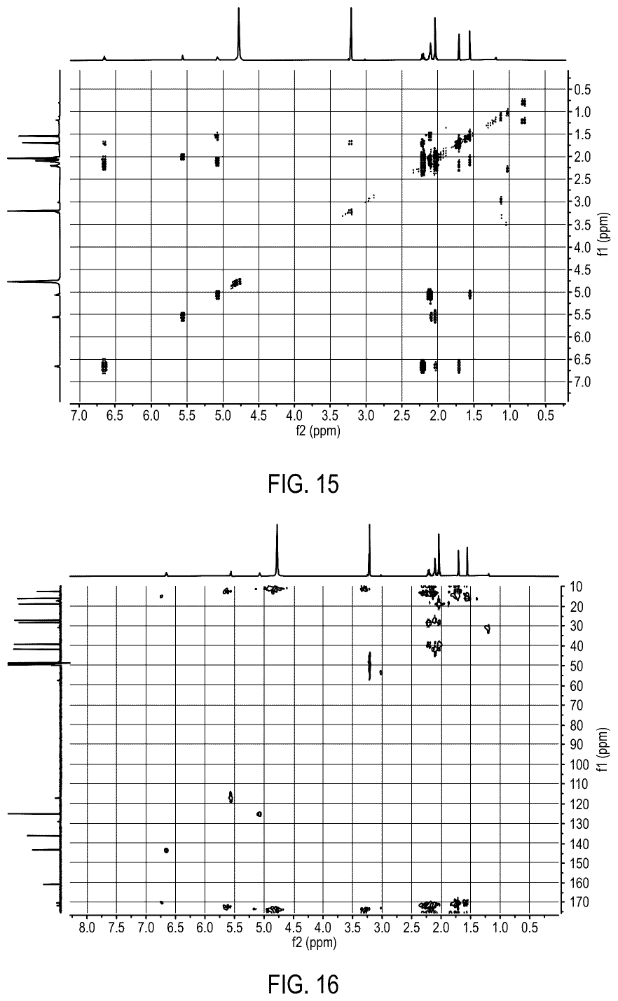

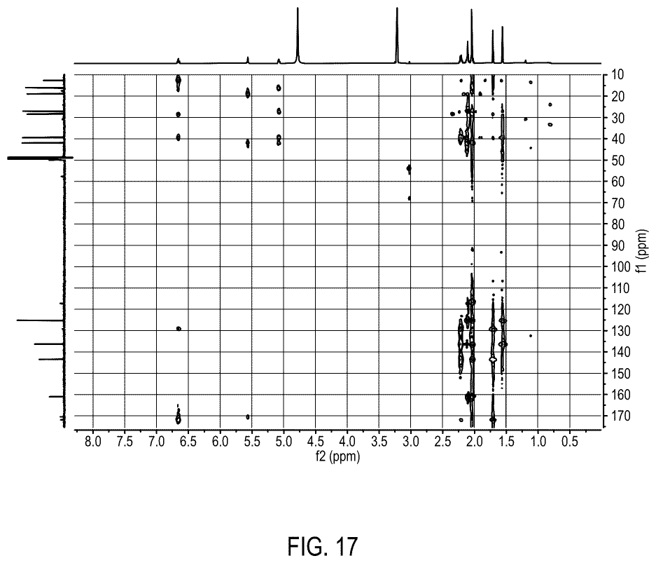

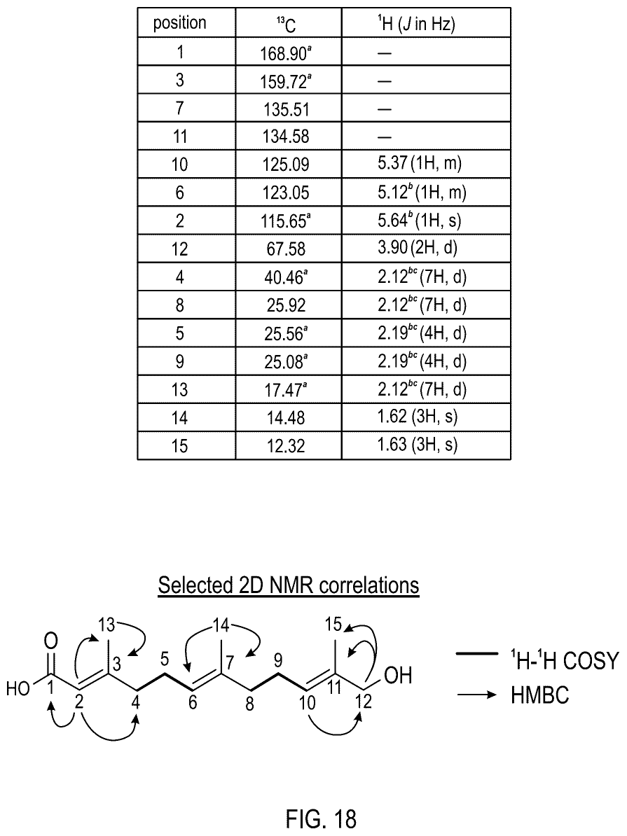

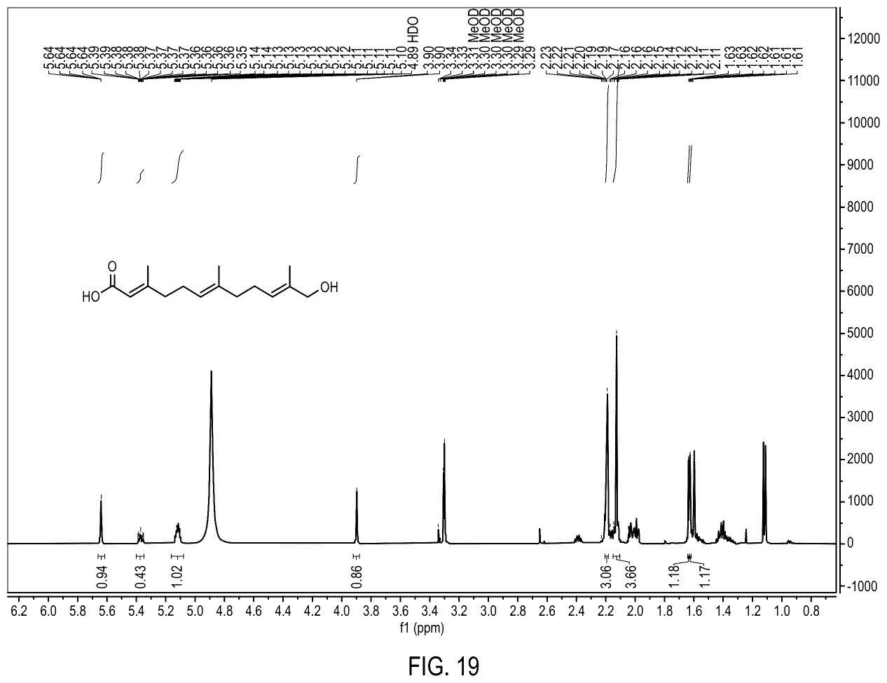

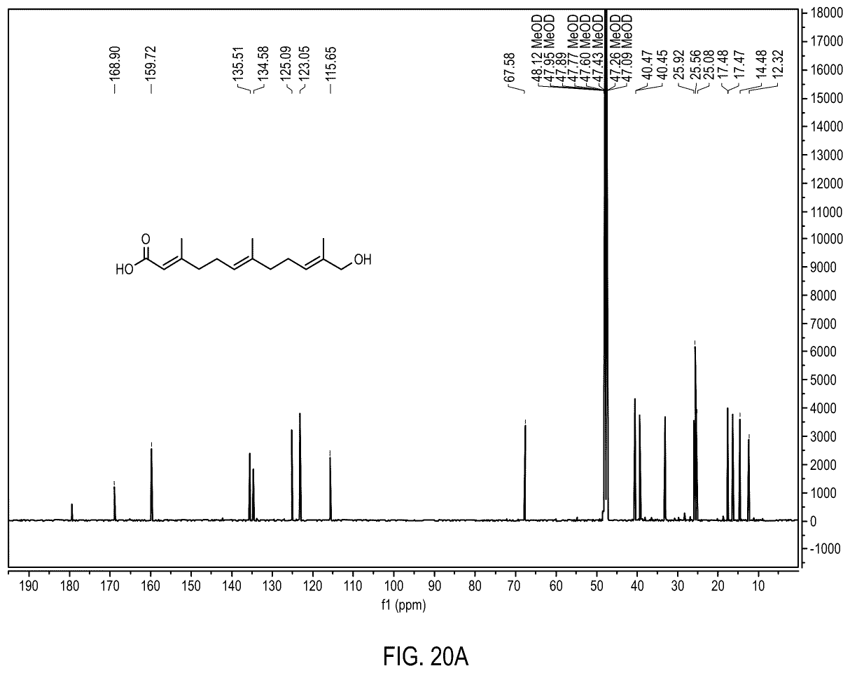

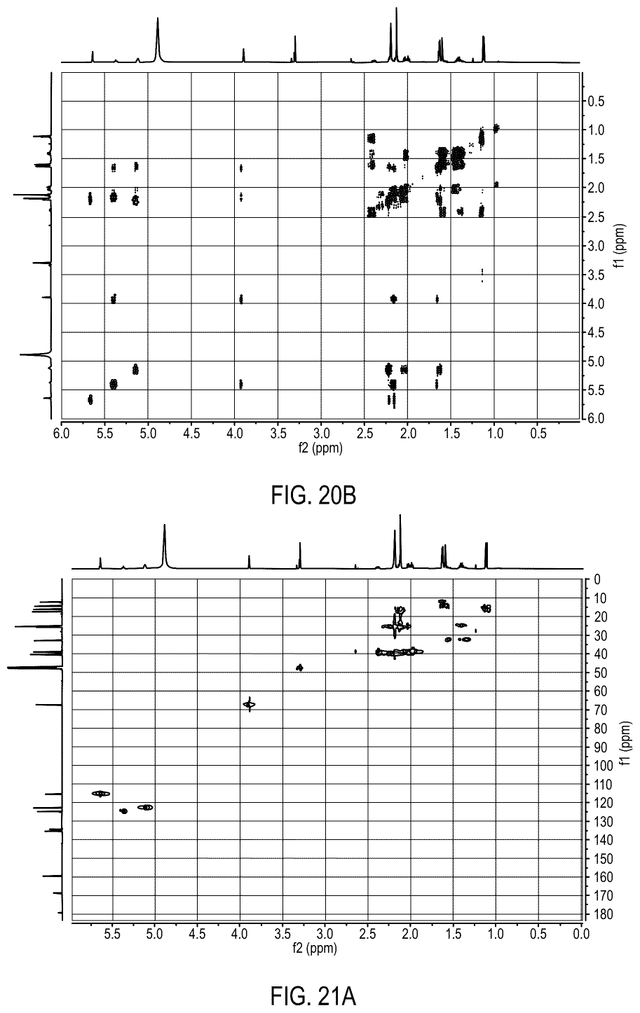

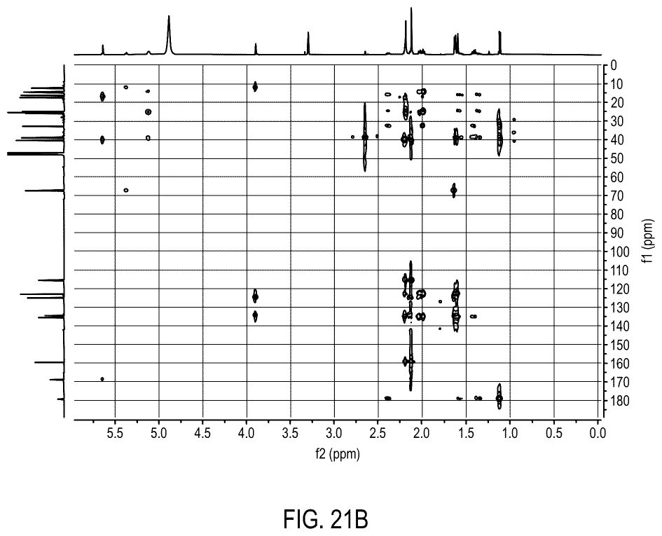

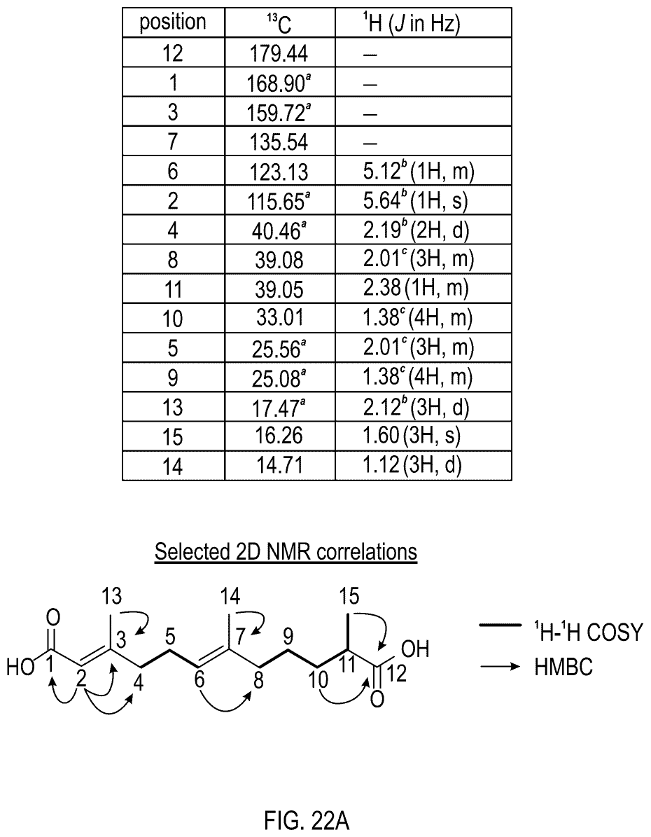

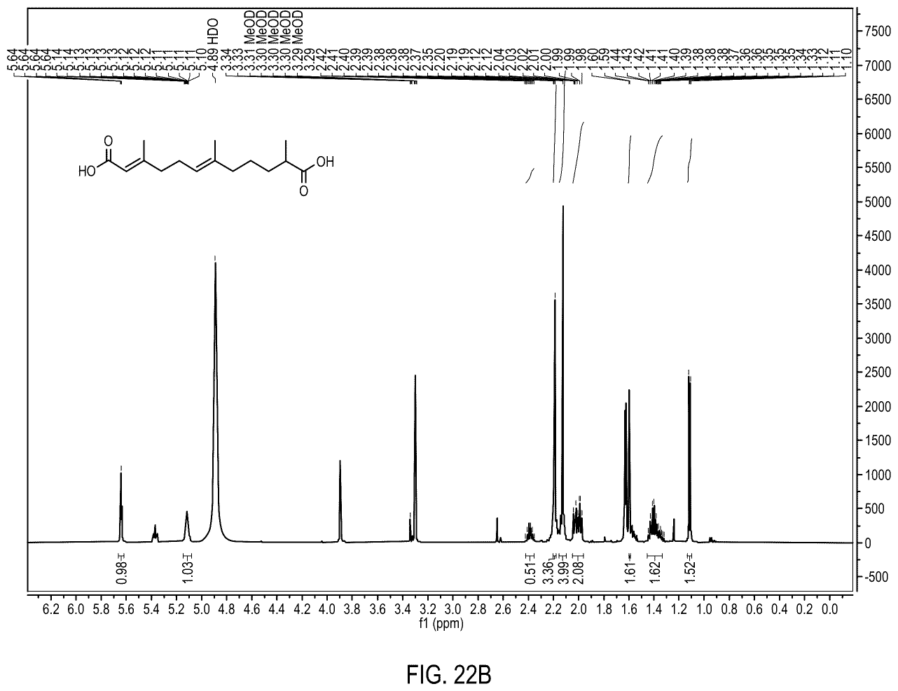

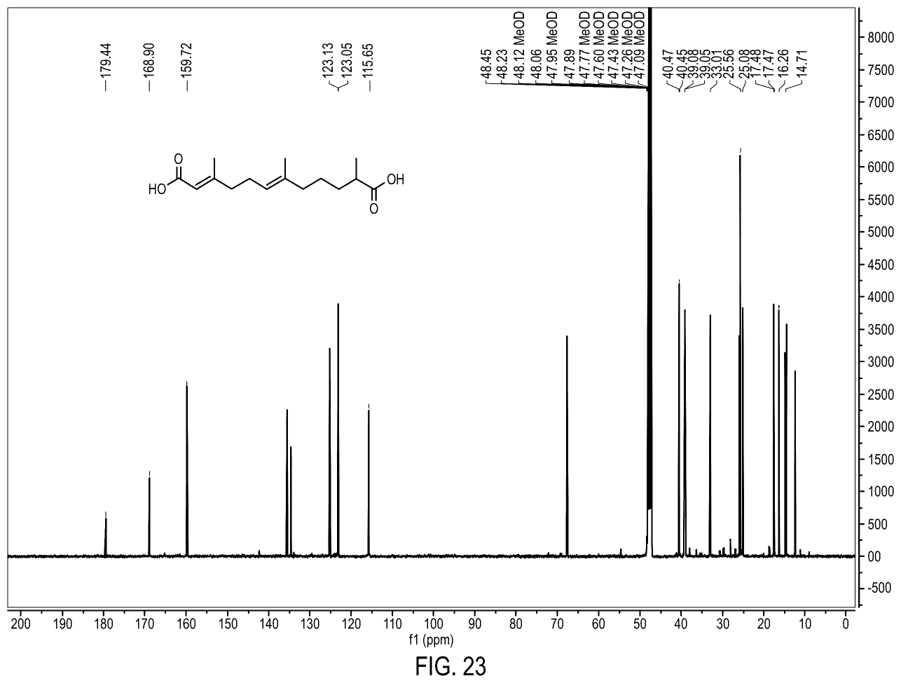

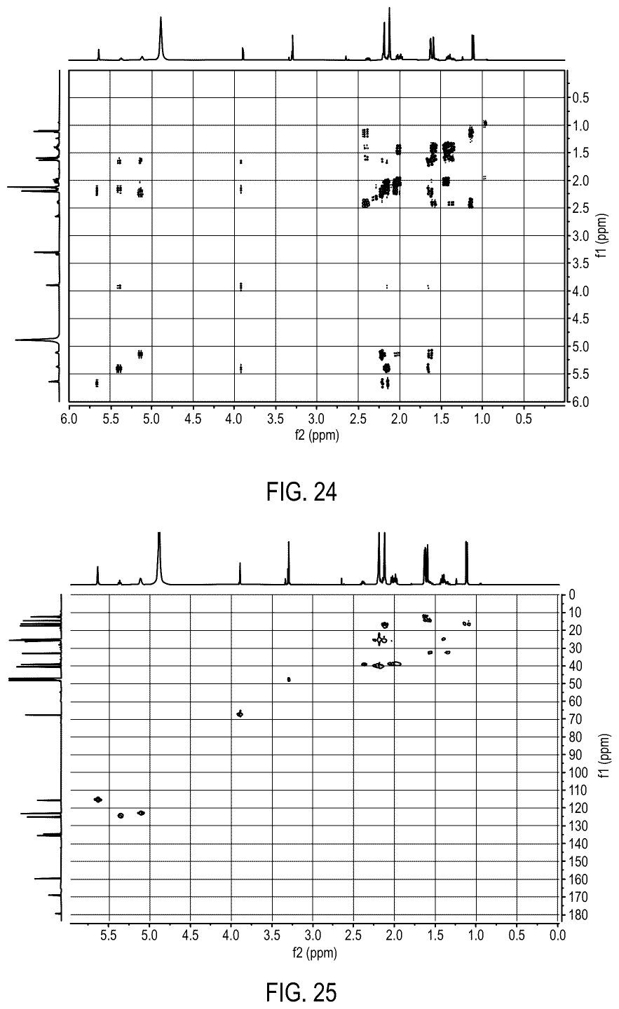

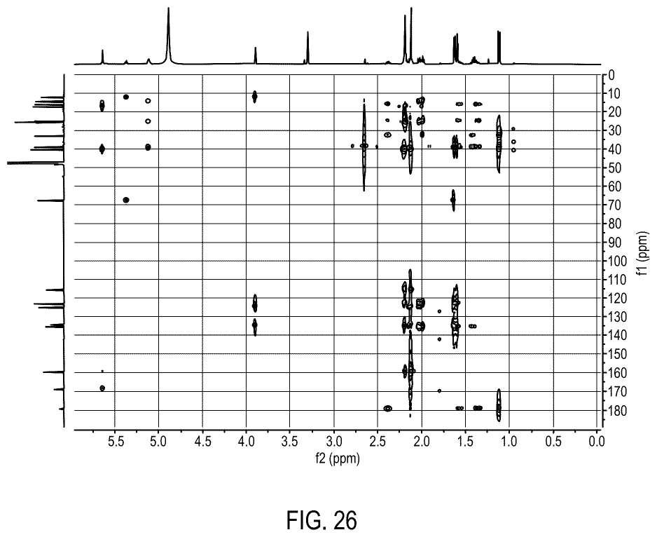

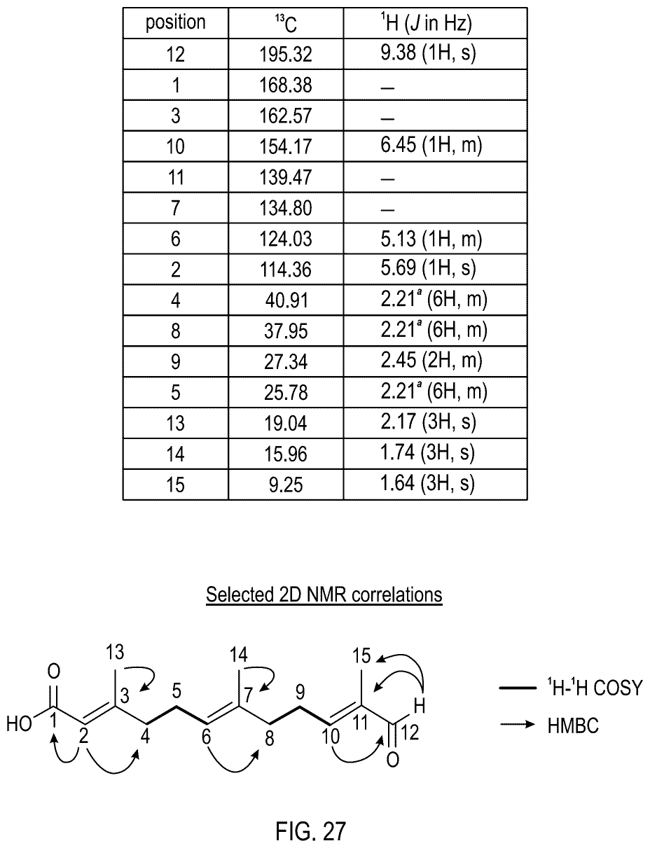

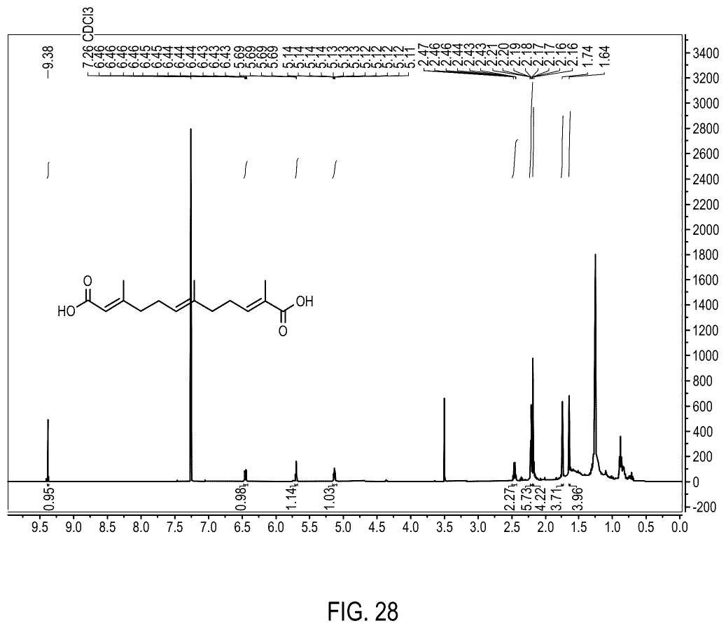

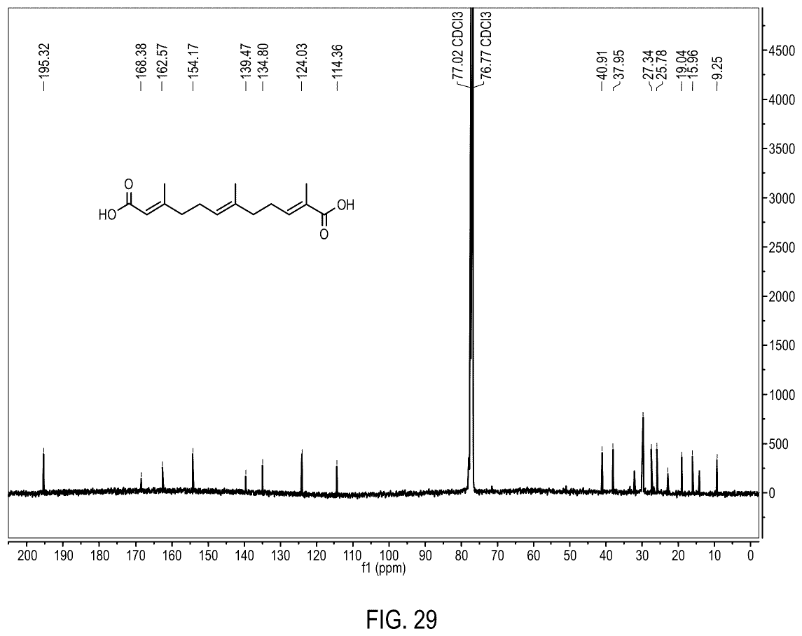

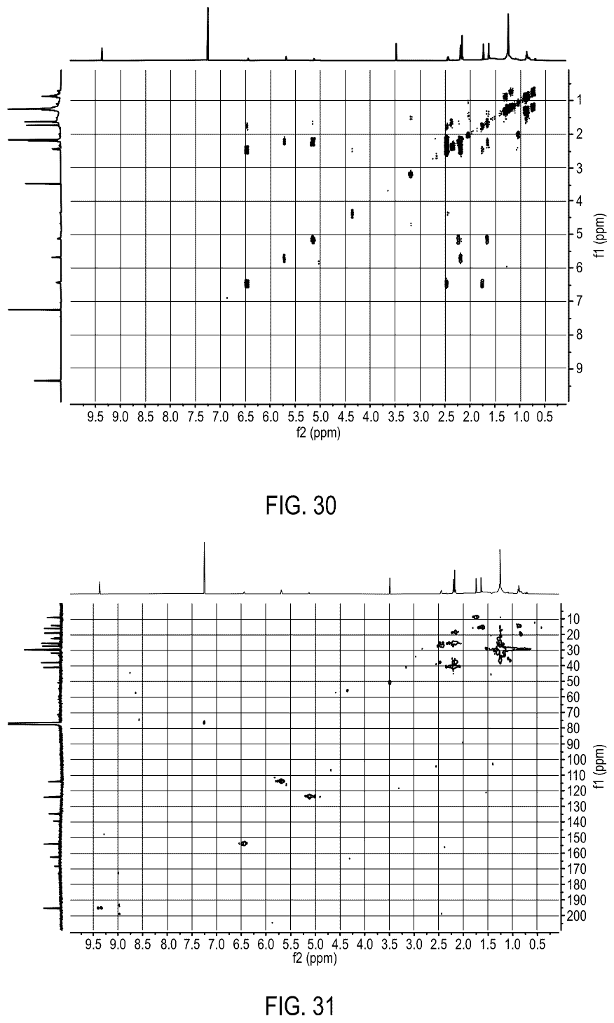

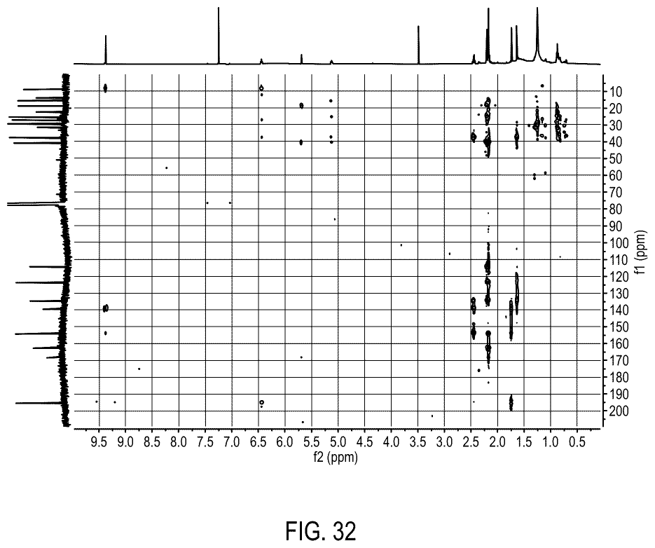

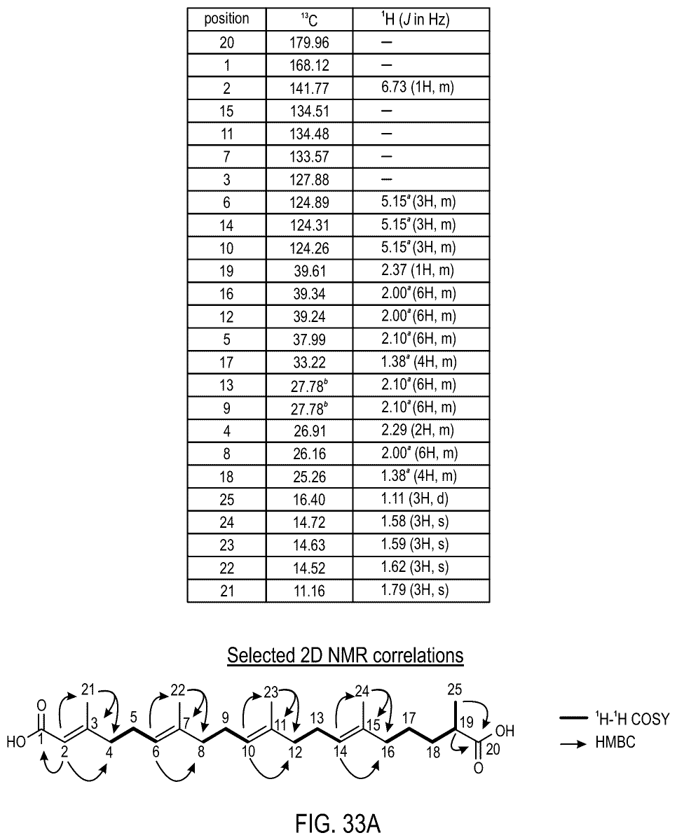

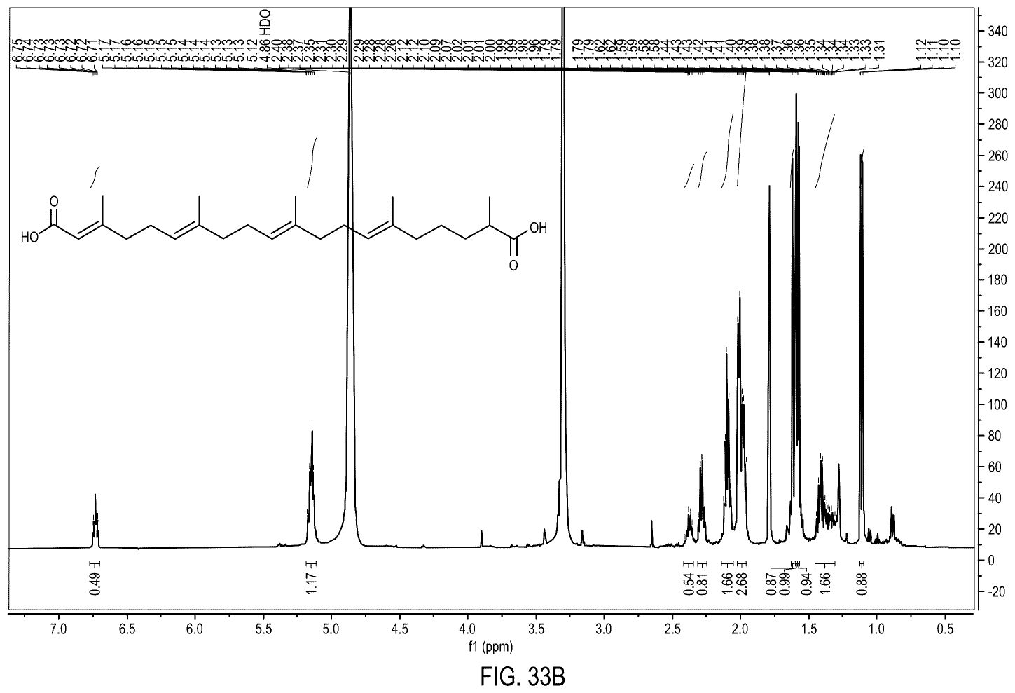

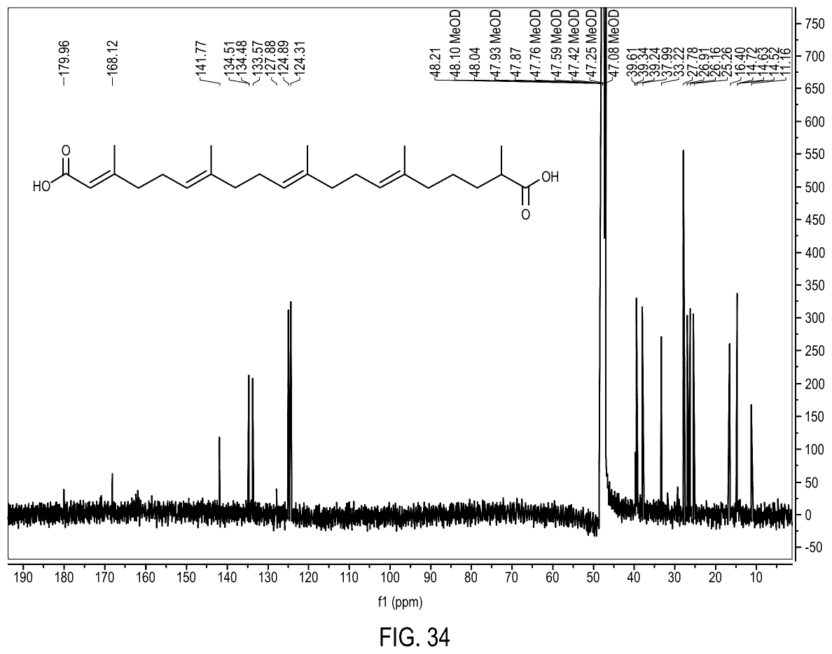

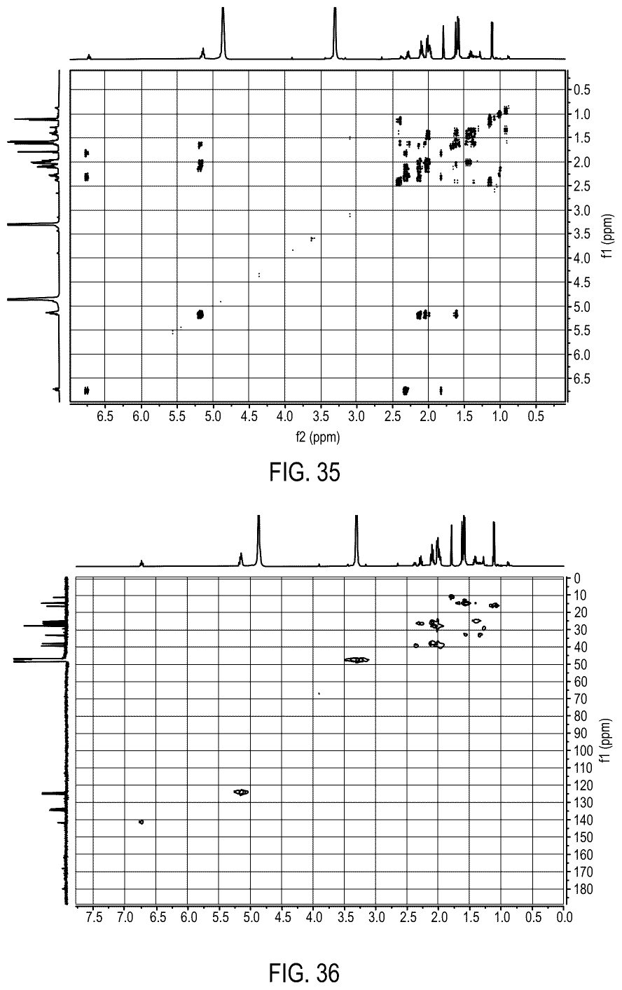

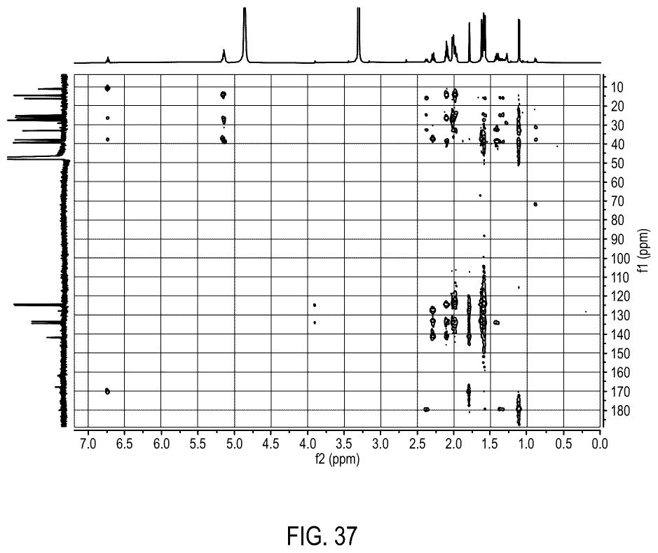

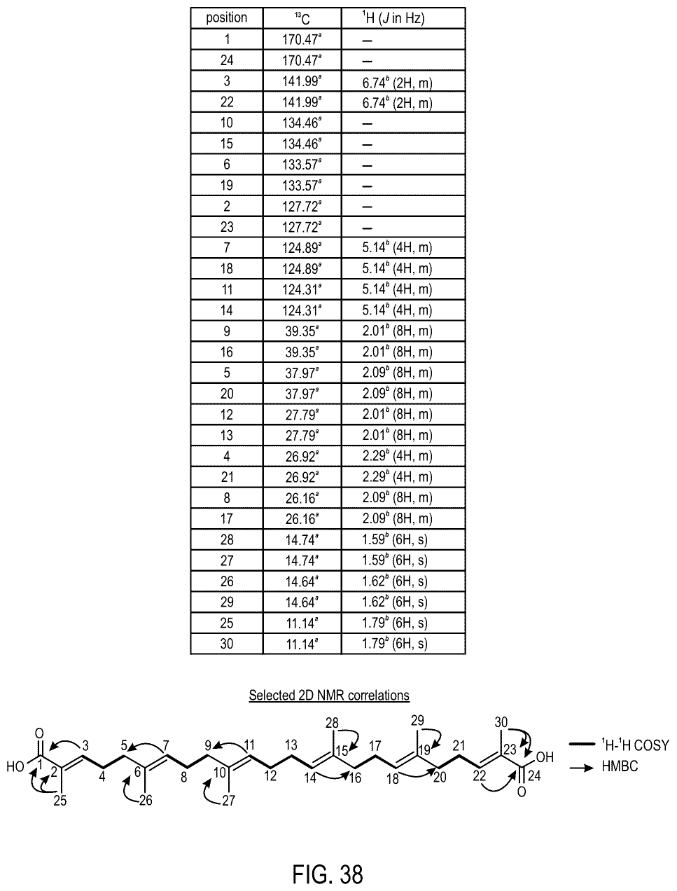

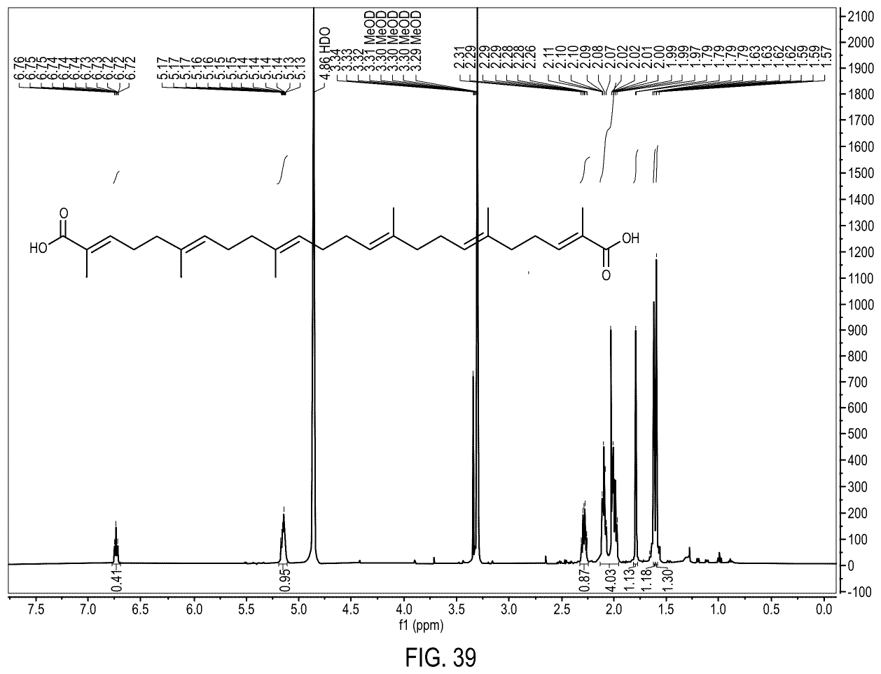

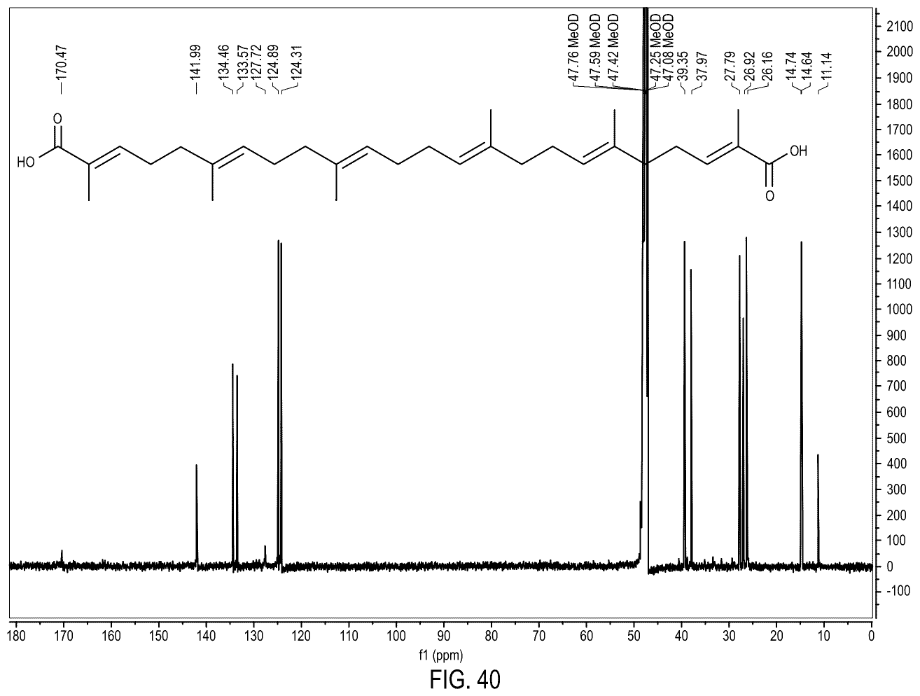

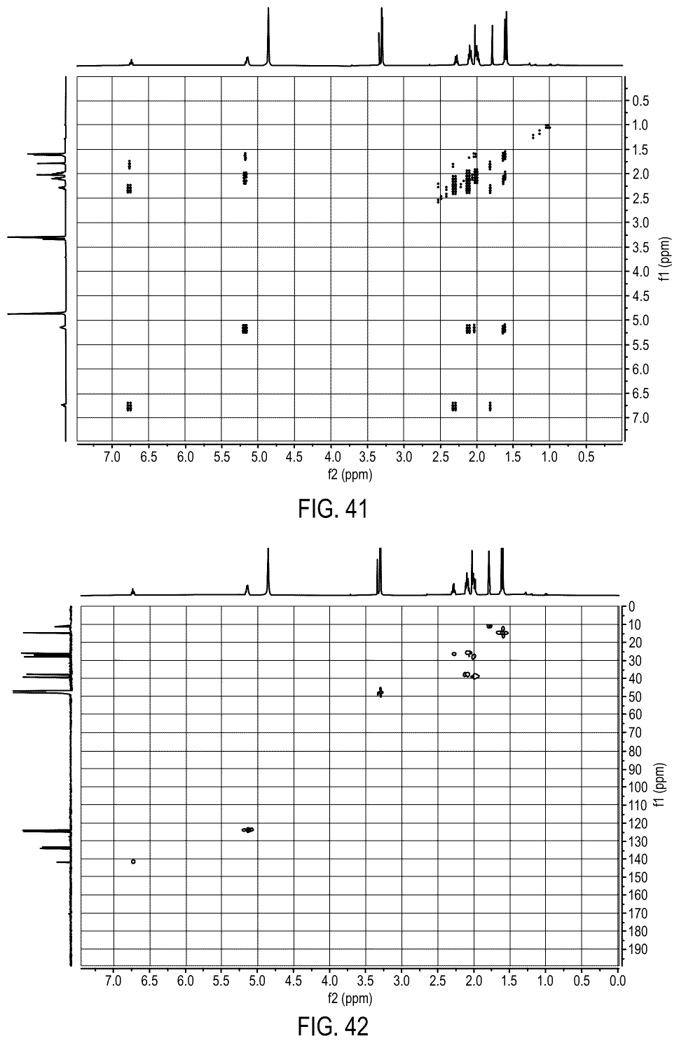

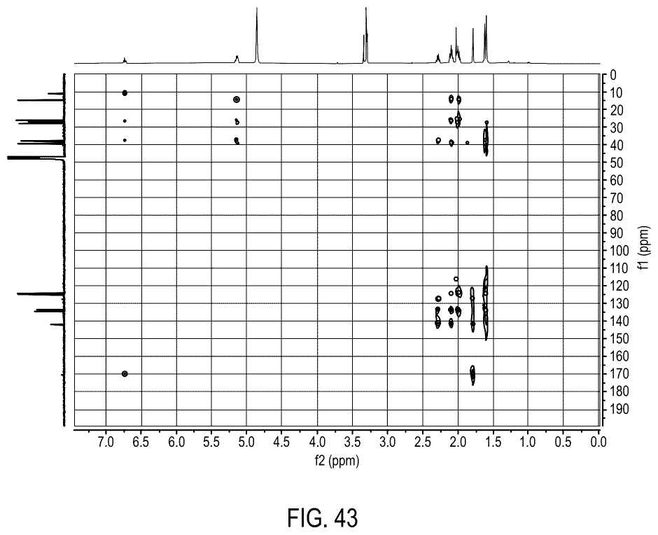

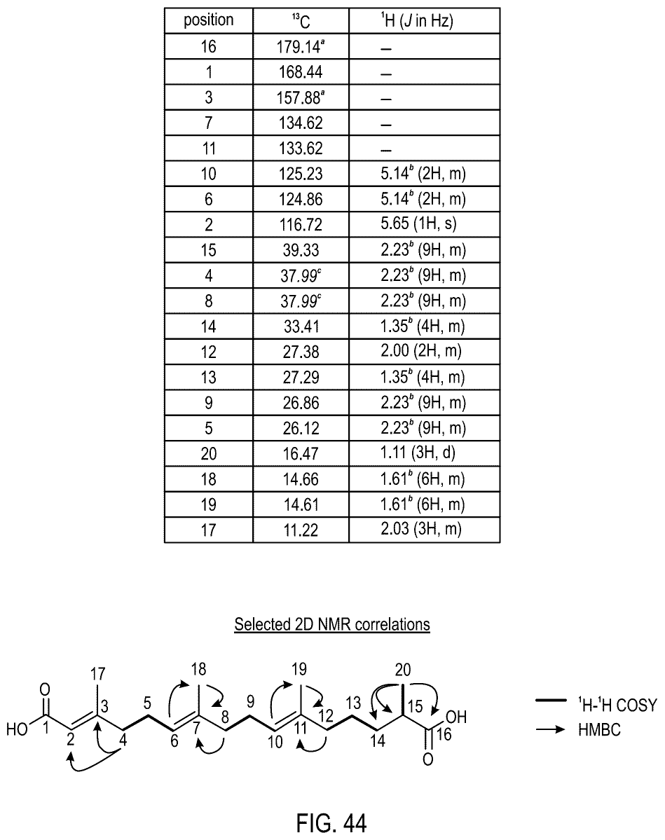

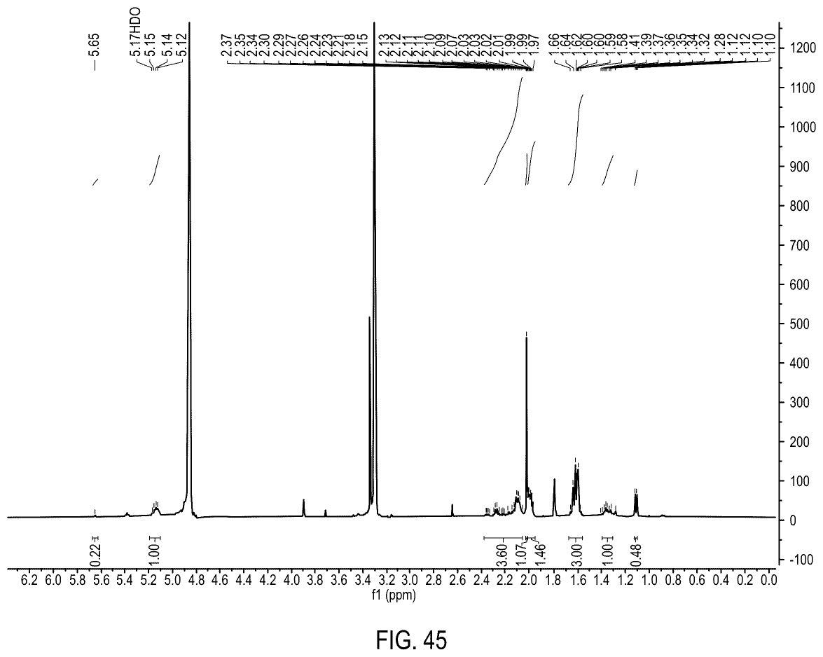

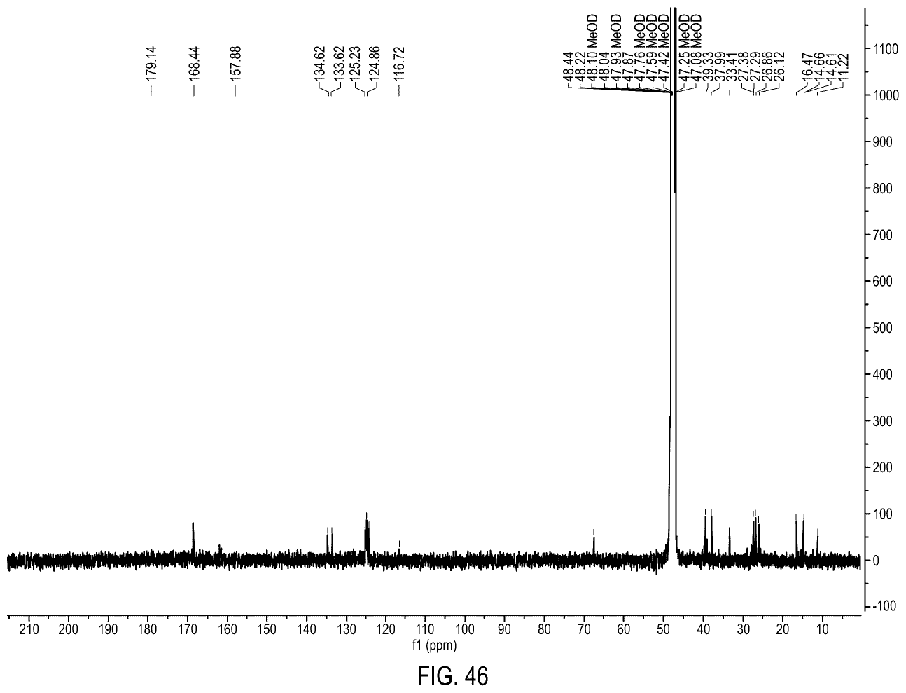

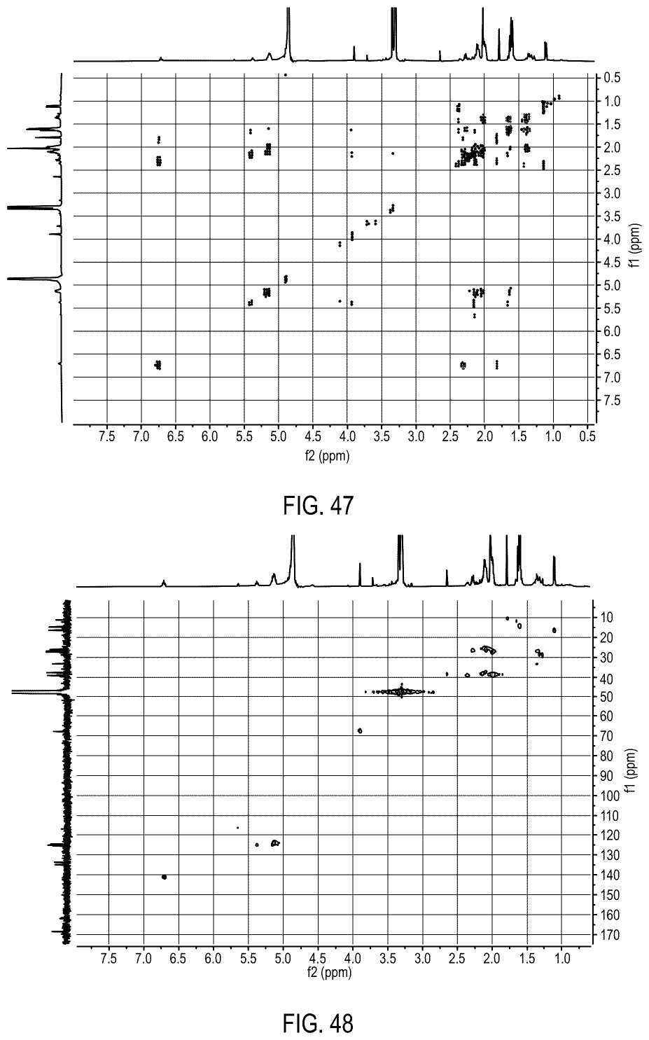

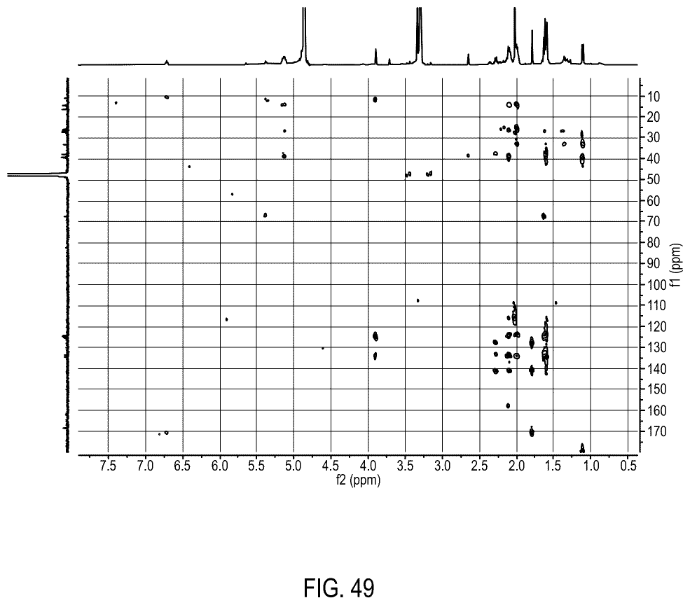

Following compound purification via culture extraction and structural elucidation through nuclear magnetic resonance (NMR) spectroscopy (FIGS. 12-49), we identified the molecule as a farnesol derivative with α- and ω-carboxylic acid functionalities, which we named farnesoic diacid (FIG. 1c). Since this compound was detected only in strains overexpressing HMGR and FPPS, but not in the wild-type, we hypothesized that elevated FPP availability redirected metabolic flux from canonical terpenoid biosynthesis towards atypical functionalization pathways.

### Identification and Structural Determination or Related Sesquiterpenoid-Derived Compounds

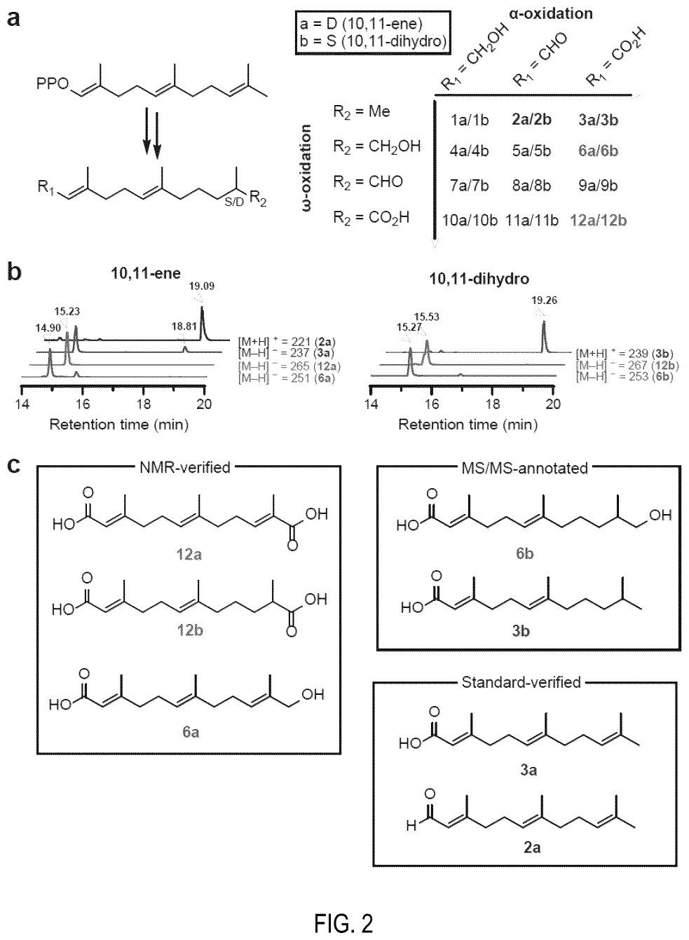

Since farnesoic diacid production resulted solely from overexpression of the mevalonate pathway, we hypothesized that increased flux toward FPP led to the activation of otherwise silent metabolic routes, where enzymes catalyzed the requisite oxidation reactions. Given the structural transformations required, we proposed that oxidation could occur at either the α- or ω-termini of the molecule to our confirmed compound farnesoic diacid (12a) (FIG. 2a). Additionally, we posited the existence of reduced molecules through the action of ene-reductase and examined our LC-MS data for evidence.

Indeed, we observed peaks corresponding to the expected m z values of several proposed intermediates (FIG. 2b). Following analysis via NMR, comparison to commercial standards, or MS/MS fragmentation, we assigned structures to these peaks (FIG. 2c, FIGS. 12-49). Notably, we detected both the 10,11-ene and the 10,11-dihydro forms of several intermediates, beginning from the α-oxidized acid intermediate (3a/3b). This suggests that its structural resemblance to fatty acids may permit recognition and reduction by ene-reductase. Together, these findings support a stepwise oxidation pathway in which the α-terminus is modified first followed by oxidation at the ω-position. However, this does preclude the other extreme route including the ω-terminus oxidation first followed by α-terminus or any routes in between. The ω-first oxidation route may be less favorable kinetically and thermodynamically, resulting in low abundance and stability of their intermediates.

### ALK Enzymes Drive Farnesoic Diacid Biosynthesis

Given that 12a was discovered through untargeted metabolomics, we could not employ the conventional genome-mining strategy to identify the associated biosynthetic genes corresponding to an NP. We reasoned that enzymes that work on aliphatic substrates would facilitate terminal oxidation. The initial oxidation step involves Cytochrome P450-mediated C—H bond activation to introduce a hydroxyl group, with the P450 enzymes belonging to the CYP52 gene family found in various fungi that can assimilate hydrophobic substrates. In Y. lipolytica, these P450s, known as ALK proteins, have been shown to oxidize fatty acids and alkanes². The final ω-terminus oxidations of fatty acids and alkanes are carried out by non-P450 oxidizing enzymes; Y. lipolytica utilizes an alcohol oxidase (AOX) and various aldehyde dehydrogenases (ALDH) 3.4. To test the biocatalytic activity of each of these enzymes, we examined their individual activity in heterologous systems by substrate-to-product conversion.

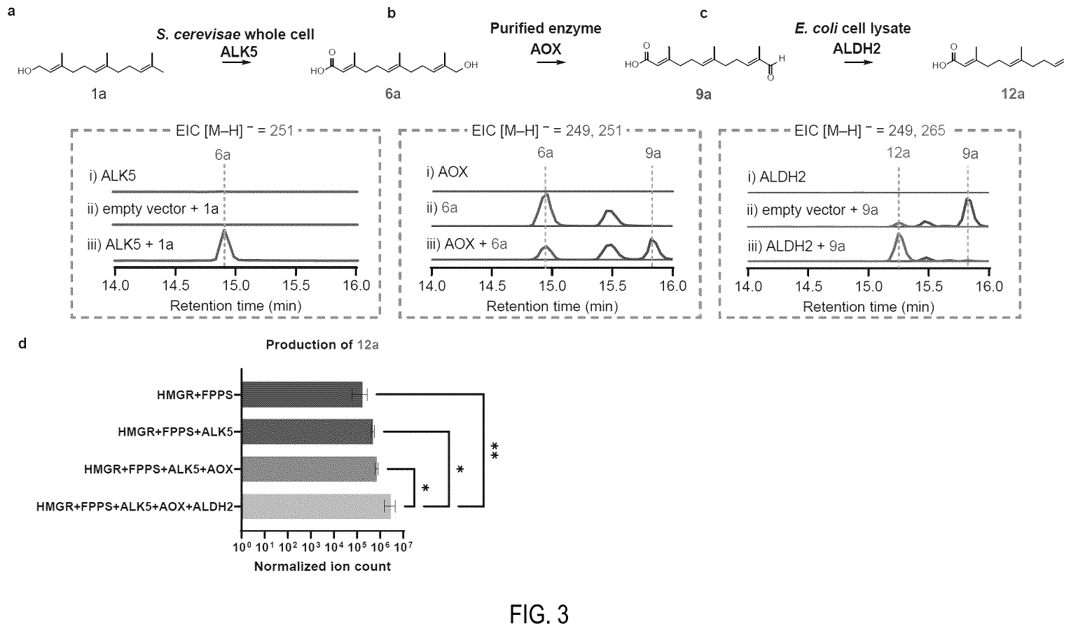

Given the challenges of in vitro P450 expression, we created a strain of S. cerevisiae with episomal expression of ALK5 for whole cell biocatalysis. The culture was grown and exchanged into a Tris buffer with or without farnesol (1a). This resulted in the emergence of previously verified compound 6a in the ALK5 with 1a reaction, but not in the empty vector and no 1a controls (FIG. 3a). Interestingly, the production of a hydroxyacid is in contrast to biocatalytic activity of a similar P450 in Mycobacterium tuberculosis, in which 1a is converted into the diol derivative⁵. This diol was not observable in any culture extracts nor was it utilized by ALK5 in biotransformation assays, suggesting a different enzymatic mechanism as it is able to regioselectively oxidize farnesol at the α-terminus first.

Next, we performed in vitro reactions with AOX expressed and purified from E. coli. This afforded a new compound 9a (MWT: 250) was observed in reactions with both 6a and AOX while absent in control reactions (FIG. 3b). Furthermore, consumption of 2a was only seen in the presence of AOX. However, 9a was not present in Y. lipolytica or S. cerevisiae culture extracts, likely due to aldehyde reactivity or toxicity preventing in vivo accumulation. Thus, we isolated and elucidated 9a from our in vitro reaction (FIGS. 12-49), which confirmed that an acid-aldehyde intermediate is formed by AOX, for which 6a is the precursor. These data support the hypothesis that there are other labile or transient intermediates that may not be detected in culture extracts but could correspond to the alternate routes proposed in FIG. 2a.

To assay the activity of ALDH2, we first attempted S. cerevisiae biotransformation. However, this resulted in background activity with aldehyde oxidation, likely due to the presence of promiscuous ALDHs in S. cerevisiae or the high reactivity of 9a (FIGS. 12-49). Attempts to express and purify ALDH2 from E. coli were also unsuccessful due to its transmembrane domain. Thus, we opted to examine ALDH2 activity in E. coli lysate to minimize background oxidation while circumventing the need to purify the enzyme. Gratifyingly, we observed formation of 12a from ALDH2-expressing lysate, with minimal formation in the empty vector control and no formation without supplementation of 9a (FIG. 3c). Furthermore, the consumption of 9a in the ALDH2-expressing lysate, but not in the empty vector lysate, confirmed 9a as the substrate for ALDH2.

With several biosynthetic enzymes identified, we then examined the impact of their overexpression in Y. lipolytica (FIG. 3d). Notably, overexpression of each enzyme increased the production of 12a compared to the base engineered strain. The additive effect observed further supports each enzyme contributes to the biosynthesis of 12a. Collectively, overexpression and characterization of each individual enzyme confirmed that ALK5, AOX, and ALDH2 catalyze the biosynthesis of 12a.

### Pathway Reconstitution and Kinetics Reveal Multifunctionality of AOX and Substrate Preference of ALK5

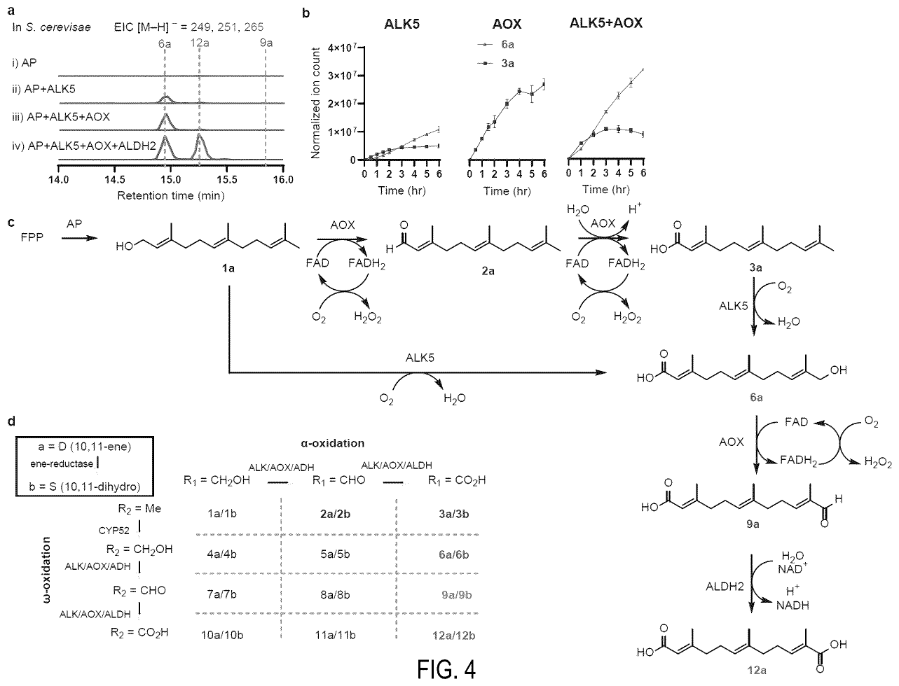

To examine the production of 12a and its precursors in a heterologous host, we reconstituted the entire biosynthetic pathway in S. cerevisiae (FIG. 4a). The genes ALK5, AOX, ALDH2, and an alkaline phosphatase (AP) to catalyze the dephosphorylation of FPP to farnesol were expressed in a stepwise fashion for metabolite analysis. As expected, production of 6a was only observed when ALK5 is expressed and no accumulation of 9a in vivo was detected even with expression AOX. Finally, expression of all four genes led to the production of 12a.

Considering the increased accumulation of 6a with AOX expression (in addition to ALK5 expression) in our in vivo reconstitution system, we hypothesized that AOX facilitates more efficient biocatalysis of 6a. To explore this, we conducted a time-course biotransformation assay (FIG. 4b). When feeding 1a to ALK5, we observed slow formation of 6a and slight accumulation of farnesoic acid (3a), suggesting its rapid conversion to 6a. In contrast, AOX supplementation with farnesol led to rapid accumulation of 3a without any production of 6a. However, when both AOX and ALK5 were presented with farnesol, we observed similarly low levels of 3a, as in the ALK5-only reaction. Instead, 6a accumulated rapidly, in contrast to the accumulation of 3a in the AOX-only reaction.

These results provide evidence that while ALK5 alone can convert farnesol to 6a through farnesoic acid as an intermediate, the initial α-oxidation step is slower than the subsequent ω-oxidation. However, AOX enables the accumulation of farnesoic acid, thus accelerating the production of 6a by ALK5. We posit that this kinetic difference is likely due to a substrate preference for more oxidized farnesol derivatives, which is further supported by biotransformation assays. Although farnesol, farnesal, and farnesoic acid were all utilized, increasing levels of production of 6a were obtained as the oxidation state of the substrate increased. While the ability of ALK5 has been shown to oxidize both alcohols and fatty acids⁷, our kinetic studies establish the differential catalytic efficiency across farnesol and its oxidized derivatives as substrates.

We elucidated the biosynthetic pathway for 12a (FIG. 4c). Following dephosphorylation of FPP by AP, there are two routes to yield 6a: either via ALK5 catalyzing the entire reaction or through AOX to generating farnesoic acid, which is subsequently utilized by ALK5. Further oxidation at the α-terminus by AOX yields the acid-aldehyde intermediate 9a, which is short-lived and oxidized by ALDH2 to afford 12a. We then updated our biosynthetic network with potential intermediates from FIG. 2a to include both confirmed biosynthetic enzymes as well as potential biosynthetic enzymes, which if overexpressed in vivo or expressed in vitro could produce intermediates otherwise challenging to detect in the extracts of present strains (FIG. 4d).

### Varied Chain-Length Diacids are Produced by Other ALK Proteins

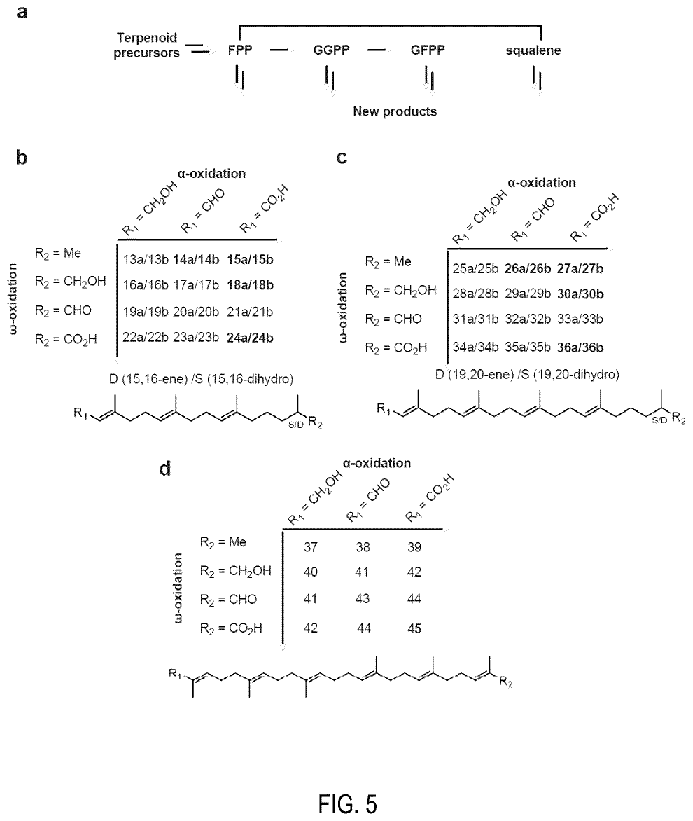

Inspired by the discovery and biosynthesis of farnesoic diacid from the result of terpenoid precursors and ALK enzymes, we looked for other similar terpenoid diacids. We hypothesized that since our strains were engineered to produce increased amounts of FPP, other terpenoid precursors such as geranylgeranyl-pyrophosphate (GGPP), geranylfarnesyl-pyrophosphate (GFPP), and squalene were also increased, as FPP is the precursor to these longer compounds (FIG. 5a). We then charted similar biosynthetic schemes, where oxidation could occur at either the α- or ω-termini of the molecule, with potential ene-reductase activity for the C20 and C25 molecules due to their similarity to C15 molecules (FIG. 5bcd).

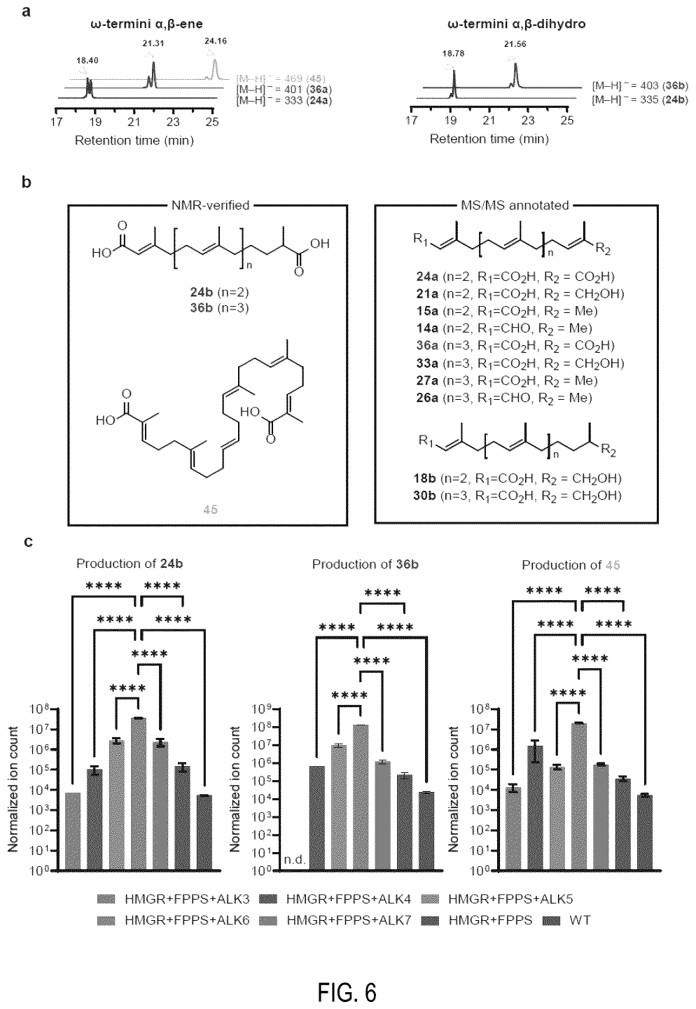

With these m z ratios corresponding to longer chain terpenoid diacids and their biosynthetic intermediates, we analyzed culture extracts with LC-MS of strains overexpressing HMGR, FPPS, and ALK proteins. Gratifyingly, we found signals corresponding to several of these proposed compounds, as the strain overexpressing HMGR+FPPS+ALK6 had more peaks not found in the WT (FIG. 6a). After isolation and NMR analysis of these compounds and MS/MS analysis of related intermediates, we found that these were indeed different-length bifunctional terpenoids, corresponding to diterpenoids, sesterterpenoids, and triterpenoids that similarly varied in the reduction or retention of the α-β double bond close to the ω-termini (FIG. 6b). There were also signals corresponding to the aldehyde, acid, and hydroxyacid compounds. Interestingly, ALK6 produced the highest amounts of the longer-chain terpenoid diacids (FIG. 6c). From these experiments, we determined that ALK6 is able to oxidize a wide variety of substrates in terms of length and form (as C30 was head-to-head connection of terpenoids).

### Bioactivity of Farnesoic Diacid and Other Bifunctional Terpenoids

Given the structural similarity between 12a and other molecules involved in cell membrane synthesis, signaling, and lipid metabolism, we investigated whether this newly identified compound exhibited bioactivity across different biological systems. We supplemented cultures with 12a to a final concentration of 10 μM and monitored growth in both Escherichia coli (FIG. 6a) and mammalian cell lines (FIG. 6b). Surprisingly, treatment with farnesoic diacid led to an increased growth rate and higher final optical density in E. coli. In contrast, a modest decrease in proliferation was observed in both D3 and Ras immortalized baby mouse kidney (iBMK) cell lines. These findings provide evidence that farnesoic diacid and potentially our other newly discovered bifunctional terpenoids may have distinct bioactivities across diverse biological contexts, with applications in more efficient cultivation of microbes and selective activity against certain mammalian cells. As the longer bifunctional terpenoids (C25-C30) resemble oxylipin and polyunsaturated fatty acids, which are implicated in immunity and obesity, we reason that bioactivity may be related to lipid metabolism in mammalian systems that will be relevant in pharmaceutical contexts. Furthermore, the shorter bifunctional terpenoids (C15-C20) and their preceding biosynthetic intermediates resemble inhibitors of insect development, such as juvenile hormones, these molecules may serve as scaffolds for insecticide drug activity to target insect development. Further research on the novel molecules will illuminate more bioactivities on humans, human cells, research animals, plants, insects, and microorganisms.

### Derivatization and Modification of Bifunctional Terpenoids

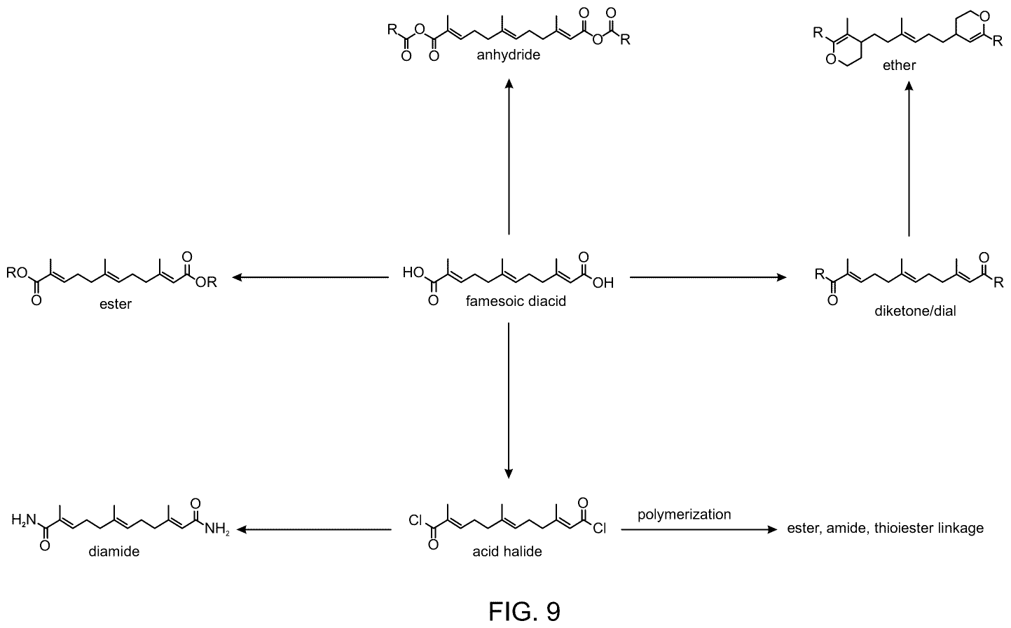

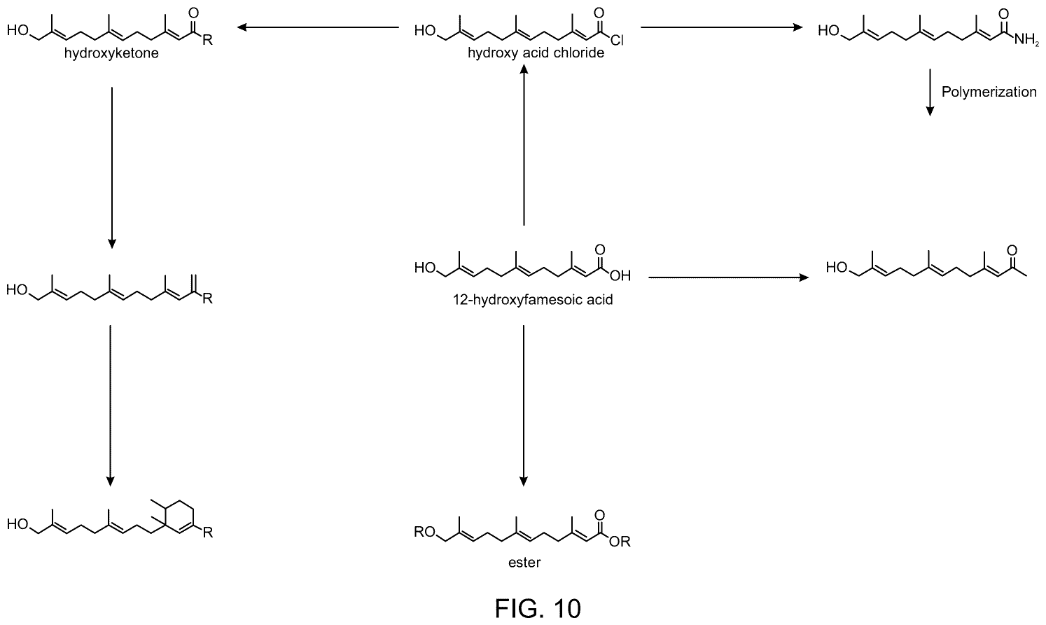

With varied lengths, desaturations, and oxidative state of functionalized termini of the discovered terpenoids, these compounds have the potential for derivatization with applications in polymer and NNP synthesis. FIGS. 9 and 10 illustrate several reaction schemes by which a diacid and a hydroxyacid may be easily derivatized to facilitate polymerization or confer reactivity for synthetic purposes. The biological origin of these molecules may be beneficial for the purpose of constructing biodegradable polymers as an alternative to fossil-fuel derived polymers. Furthermore, as acyclic bifunctionalized compounds can serve as privilege scaffolds for the construction of complex (N)NPs10, this research represents a sustainable and scalable source of these molecules for renewable synthesis.

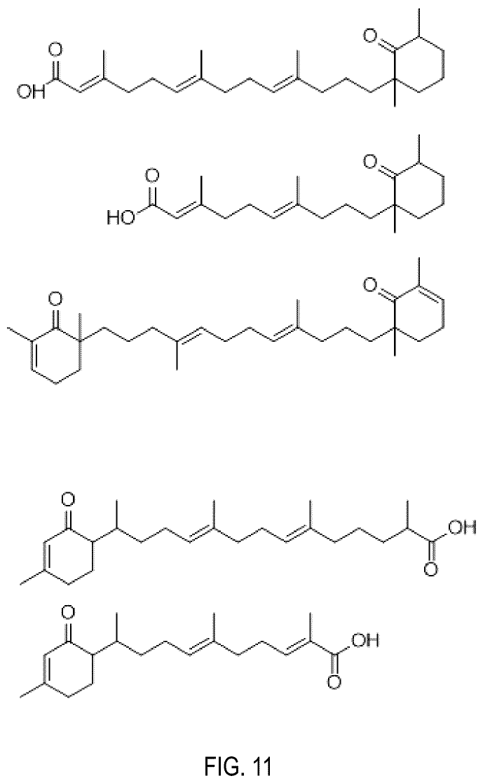

Additionally, due to the potentially reactive nature of the diacid ends of the newly discovered bifunctional terpenoids, we hypothesized that several annulations could occur either enzymatically or synthetically (FIG. 11). At the ω-termini, we hypothesized that a cyclic ketone could arise from the π-electrons of carbon 2 of the preceding isoprenyl unit (FIG. 11). Similarly, a cyclic ketone could be created at the α-termini though the π-electrons of carbon 3 of the following isoprenyl unit. Thus, these compounds could be used by themselves or in combination with linear diacids for various applications. Further research will be conducted to maximize the production of the novel bifunctional terpenoids for increased access to these molecules, facilitating polymerization studies for material characterization and synthesis efforts.

### Methods

### Materials

Biological reagents, chemicals, and media were purchased from standard commercial sources unless stated. DNA isolation was carried out using Zymo Quick-DNA Fungal/Bacterial Kit. High-fidelity PCR amplification was carried out using NEB Q5 Hot Start High-Fidelity Master Mix, and colony PCR screening was carried out with Thermo Scientific Phire Tissue Direct PCR Master Mix. DNA sequencing was performed at Plasmidsaurus. The primers were synthesized by IDT and the codon-optimized genes were synthesized by Twist Bioscience.

Integration and Overexpression of Genes in Y. lipolytica

To overexpress the mevalonate or sesquiterpenoid pathway or identify potential biosynthetic enzymes for farnesoic diacid, plasmids pHR_AXP_hrGFP and pHR_D17_hrGFP⁸ were digested with NheI and AscI to excise hrGFP and insert genes or expression cassette elements. Promoters (pFBAin, pEXP), terminators (LIP2t, PRC1t), and genes to be expressed were generated by PCR amplification with the genomic DNA of Y. lipolytica PO1f as the template. The recombinant pHR plasmids were generated using NEBuilder HiFi DNA Assembly (NEB) with the digested vector backbone and inserts with 30 bp homology regions, which were immediately transformed into NEB5-alpha Competent E. coli for sequencing verification.

Recombinant pHR plasmids were co-transformed with their corresponding pCRISPRyl plasmids into Y. lipolytica using Frozen-EZ Yeast Transformation II Kit (Zymo Research). Selection for cassette integration and plasmid dropout were performed as described by Schwartz et al⁸.

Construction of S. cerevisiae Strains

For episomal heterologous expression, plasmids pXW55 (URA3 marker), pXW06 (TRP1 marker), and pXW02 (LEU2 marker) digested with NdeI and PmeI were used to introduce the candidate Y. lipolytica genes. The recombinant pX plasmids were generated using NEBuilder HiFi DNA Assembly (NEB) with the digested vector backbone and inserts with 30 bp homology regions, which were immediately transformed into NEB5-alpha Competent E. coli for sequencing verification. Recombinant pX plasmids were transformed into S. cerevisiae RC01 to generate the strains.

### Metabolite Extraction

To measure intracellular metabolites and measure the perturbed metabolism after genetic manipulations on Y. lipolytica, 1 ml of culture was vacuum-filtered on membrane filters (nylon, 47 mm diameter, 0.45 μm pore size, Millipore) and flipped cell-side down into 400 μl of extraction solvent (40:40:20 acetonitrile/methanol/water) in a six-well plate. To quickly quench metabolism and minimize metabolite degradation, filtration was performed as quickly as possible, and the extraction solvent was pre-cooled to −20° C. After incubation at −20° C. for 20 minutes, the filter was flipped cell-side up and washed with the extraction solvent in the well. The extract was then collected in a 1.5 ml centrifuge tube and centrifuged at 4° C. The supernatant was then used directly for LC-MS analysis.

### Metabolite Measurement and Characterization

LC-MS analysis was carried out on an HPLC (Vanquish Duo UHPLC, Thermo Fisher Scientific) coupled to a high-resolution orbitrap mass spectrometer (Q Exactive Plus, Thermo Fisher Scientific). The LC separation was achieved using a hydrophilic interaction chromatography column (XBridge BEH Amide XP Column, 130 Å, 2.5 μm, 2.1 mm×150 mm, Waters). MS was performed in both positive and negative mode using a mass resolution of 140,000 at 200 m/z. The resulting LC-MS data were processed using the Metabolomic Analysis and Visualization Engine (MAVEN) 9. NMR spectra were acquired on Bruker AV500 spectrometer with 5 mm dual cryoprobe (1H 500 MHz, 13C 125 MHz). The resulting spectra were processed using Bruker TopSpin and Mnova NMR software.

### Fermentation and Compound Isolation

A seed culture of Y. lipolytica strain was grown in 3 ml of YPD (yeast extract 10 g/l, peptone 20 g/1, dextrose 20 g/l) overnight at 30° C. and 350 rpm. Fermentation of the yeast was carried out using either YPD or modified YPD (yeast extract 10 g/l, peptone 20 g/l, dextrose 100 g/l, leucine 10 g/l) for 3 days at 30° C. and 250 rpm.

To isolate compound 12a, the fermentation broth of Y. lipolytica strain HMGR+FPPS was centrifuged (5,000 g, 30 min), and the cell pellet was collected and soaked in acetone while the supernatant was extracted three times with ethyl acetate. The extract was then dried and concentrated using rotary evaporation and subjected to flash silica column purification with a gradient of hexane and ethyl acetate. Fractions were tested on the HPLC-MS system described above, and those containing compound 12a were pooled and dried using rotary evaporation. The residue was dissolved in acetonitrile and subjected to semipreparative HPLC purification using a reverse-phase column (XBridge BEH C18 OBD Prep Column, 130 Å, 5 μm, 10 mm×250 mm).

To isolate compound 6a and 12b, the fermentation broth of Y. lipolytica strain HMGR+FPPS was centrifuged (5,000 g, 30 min), and the cell pellet was collected and soaked in acetone while the supernatant was extracted three times with dichloromethane. The extract was then dried and concentrated using rotary evaporation and subjected to flash silica column purification with a gradient of hexane and ethyl acetate. Fractions were tested on the HPLC-MS system described above, and those containing compound 2 were pooled and dried using rotary evaporation. The residue was dissolved in acetonitrile and subjected to semipreparative HPLC purification using a reverse-phase column (XBridge BEH C18 OBD Prep Column, 130 Å, 5 μm, 10 mm×250 mm).

To isolate compounds 24b, 36b, and 45, the fermentation broth Y. lipolytica strain HMGR+FPPS+ALK6 was centrifuged (5,000 g, 30 min), and the cell pellet was collected and soaked in 1:1 acetone: dichloromethane. The extract was then dried and concentrated using rotary evaporation and subjected to flash silica column purification with a gradient of hexane and ethyl acetate. Fractions were tested on the HPLC-MS system described above, and those containing the compound were pooled and dried using rotary evaporation. The residue was dissolved in acetonitrile and subjected to semipreparative HPLC purification using a reverse-phase column (XBridge BEH C18 OBD Prep Column, 130 Å, 5 μm, 10 mm×250 mm).

### Protein Expression, Purification and Biochemical Assay

The cDNA sequence of AOX was codon-optimized for E. coli and cloned into a pET28a plasmid with a N-(His)₆-SUMO tag using NEBuilder HiFi DNA Assembly Master Mix (NEB). The resulting recombinant pET28a plasmid was transformed into chemically competent BL21 (DE3) E. coli (NEB) and sequence verified. For protein production, a seed culture of the E. coli strain was grown in 3 ml of LB medium with 50 μg/mL kanamycin overnight at 37° C. and 250 rpm. A 1:1000 dilution of the culture was used to inoculate 1 L of LB medium with 50 μg/mL kanamycin pre-warmed at 37° C. The culture was shaken at 37° C. and 250 rpm until OD600 reached ˜0.8-1 and cooled to 16° C. IPTG was added to the cooled culture to a final concentration of 100 μM and the culture was left to induce at 16° C. and 220 rpm for 18-24 hours. After protein expression was completed, cells were harvested by centrifugation and frozen.

### Feeding Assay

To test in vivo biocatalytic activity of candidate enzymes, recombinant S. cerevisiae harboring a pX expression plasmid was utilized. A seed culture of the S. cerevisiae was grown in selective synthetic complete media overnight before fermentation of the yeast in YPD for 2 days. Cells were harvested at centrifuged at 4° C., washed with 10 mM Tris-HCl 1 mM EDTA buffer at pH 8, and resuspended in 2 mL of Tris-HCl-EDTA buffer. Proposed substrates were then spiked from a 10 mM solution in ethanol for a final concentration of 20 μM. Cultures were incubated at 30° C. while shaking at 350 rpm with shaking for an additional day. For extractions, all 3 ml of culture were extracted one time with an equal volume of ethyl acetate with 500-700 μm glass beads. The organic phase was dried and concentrated to oil form, then reconstituted in 200 ul methanol for LC-MS analysis.

### Table a: Illustrative Polypeptide Sequences

Embodiments of the invention include microorganisms engineered to express a polypeptide having at least 60%, 70%, 80%, 85%, 90%, 95%, 97%, 98% or 99% sequence identity with the amino acid sequence set out below.

β-Hydroxy β-methylglutaryl-CoA reductase:
(SEQ ID NO: 1)
MLQAAIGKIVGFAVNRPIHTVVLTSIVASTAYLAILDIAIPGFEGTQPISYYHPAAK
SYDNPADWTHIAEADIPSDAYRLAFAQIRVSDVQGGEAPTIPGAVAVSDLDHRIV
MDYKQWAPWTASNEQIASENHIWKHSFKDHVAFSWIKWFRWAYLRLSTLIQGA
DNFDIAVVALGYLAMHYTFFSLFRSMRKVGSHFWLASMALVSSTFAFLLAVVAS
SSLGYRPSMITMSEGLPFLVVAIGFDRKVNLASEVLTSKSSQLAPMVQVITKIASK
ALFEYSLEVAALFAGAYTGVPRLSQFCFLSAWILIFDYMFLLTFYSAVLAIKFEIN
HIKRNRMIQDALKEDGVSAAVAEKVADSSPDAKLDRKSDVSLFGASGAIAVFKIF
MVLGFLGLNLINLTAIPHLGKAAAAAQSVTPITLSPELLHAIPASVPVVVTFVPSV
VYEHSQLILQLEDALTTFLAACSKTIGDPVISKYIFLCLMVSTALNVYLFGATREV
VRTQSVKVVEKHVPIVIEKPSEKEEDTSSEDSIELTVGKQPKPVTETRSLDDLEAI
MKAGKTKLLEDHEVVKLSLEGKLPLYALEKQLGDNTRAVGIRRSIISQQSNTKTL
ETSKLPYLHYDYDRVFGACCENVIGYMPLPVGVAGPMNIDGKNYHIPMATTEGC
LVASTMRGCKAINAGGGVTTVLTQDGMTRGPCVSFPSLKRAGAAKIWLDSEEG
LKSMRKAFNSTSRFARLQSLHSTLAGNLLFIRFRTTTGDAMGMNMISKGVEHSL
AVMVKEYGFPDMDIVSVSGNYCTDKKPAAINWIEGRGKSVVAEATIPAHIVKSV
LKSEVDALVELNISKNLIGSAMAGSVGGFNAHAANLVTAIYLATGQDPAQNVES
SNCITLMSNVDGNLLISVSMPSIEVGTIGGGTILEPQGAMLEMLGVRGPHIETPGA
NAQQLARIIASGVLAAELSLCSALAAGHLVQSHMTHNRSQAPTPAKQSQADLQR
LQNGSNICIRS
Farnesyl pyrophosphate synthase:
(SEQ ID NO: 2)
MSKAKFESVFPRISEELVQLLRDEGLPQDAVQWFSDSLQYNCVGGKLNRGLSVV
DTYQLLTGKKELDDEEYYRLALLGWLIELLQAFFLVSDDIMDESKTRRGQPCWY
LKPKVGMIAINDAFMLESGIYILLKKHFRQEKYYIDLVELFHDISFKTELGQLVDL
LTAPEDEVDLNRFSLDKHSFIVRYKTAYYSFYLPVVLAMYVAGITNPKDLQQAM
DVLIPLGEYFQVQDDYLDNFGDPEFIGKIGTDIQDNKCSWLVNKALQKATPEQRQ
ILEDNYGVKDKSKELVIKKLYDDMKIEQDYLDYEEEVVGDIKKKIEQVDESRGF
KKEVLNAFLAKIYKRQK
ALK3:
(SEQ ID NO: 3)
MIIIETLIGAVVFVAVYVAFVKLDYYRRKAKFETSDMPVAYNGLLGWKGLRHM
LTVFNNDIGPVGWREVFATYGKTLKYYAFPSNTILTYDPDNIKAMLATQFKDFSL
GLRKEALAPSLGYGIFTLDGSSWSHSRALLRPQFSREQISRLESVETHVQEMMSCI
DRNQGAYFDIQRLFFSLAMDTATDFLLGEAVGNLQEILHPEMPRTGTTFQVAFD
RAQRLGSLRIICQEAFWVVGSLFWRRDFNNTNQHIHDYVDRYVDKALLARKEKS
EIYTNPDKYIFLYELARETTNKITLRDQVLNILIAGRDTTASTLSWIFMELAKKPDI
FHKLREAILNDFGTSCESISFESLKKCDYLRQVLNEGLRLHPVVPVNLRVAVRDT
TLPRGGGPQGDKPIFVAKGQKINYAIFWTHRDKEYWGEDAEEFRPERWETTSGG
ALGKGWEFLPFNGGPRICLGQQFALTEMGYVITRLLQEYSDISIQPSDAAVKVRH
SLTMCSAQGINISLTRAKEE
ALK4:
(SEQ ID NO: 4)
MLTNLTIVLITLLVTYTVLTRTALRIQRARKAKQMGATLPPRVNNGILGWYGLW
LVIQNARSMKLPHTLGKRFANGPTWLTPVAGNEPINTIDPENVKAILATQFKDFC
LGIRHRALSPSIGDGIFTLDGEGWTHSRALLRPQFSRQQISRVHSLERLMQILFKLI
RKENGEYFDLQNLFFMFTLDSATEFLYGASVDTLADLLGEPVEGDHGGVGEEVR
KAYQQSINNAQDISAIRTRLQGLYWIAGNIYQRNLYQKSNKGVKDFSQFFVDKA
LNTSKEKLKEMEDSDNYVFLYELVKSTRNPVVIRDQLINILVAGRDTTASLLSFTF
YTLGRRPDVLKKLRAAILEDFGTSPDEITFESLKRCDYLRYVLNEVLRLYPSVPIN
ARSATRDTTLPRGGGPDGKQPVFVYKGQMVAYCVYWMHRDKKYWGEDALEF
NPDRWDPKVQPQNKGWEYLPFNGGPRICLGQQFALTEAGYVVTRMLQEFDTVH
CKNQKEEEHPPYALDLTMRHGEGVWVSMK
ALK5:
(SEQ ID NO: 5)
MLQLFGVLVLALTTALLAQLAYNKYEYNRKVKQFGCGELTVAKNGFLGWKGIR
AVLHVLKTKKGPAALKERIDAYGRTYVFHIGPAPVISTMEPENIKAMLATQFKDF
SLGTRYRSLAPTLGDGIFTLDGHGWTHSRALLRPQFAREQVSRLDSLEAHFQILK
MCVDKEMREKGNDPRGFDIQNLFFLFTLDSATEFLFGSSVDSLVDFLDDPSVRTG
DHGGVDEAARKGFNNSFNHAQELCALRSRLHTLYWIVGSVVKKEPFERYNKEIK
TFVDFFAAKALKARKEKDMSLMDNDQYIFMYELVKETTNPVTLRDQMLNILLA
GRDTTASMLSWIYFRLARDPKLYAKLRSAILEDFGTTPEAITFESLKQCDYLRYV
LNEALRLYPVVPINGRTATRDTTLPRGGGPDQSQPIFIPKGQTVSYSVYWTHRDP
RFWGEDAEEFIPERWDPRNGNIGRGWEYLPFNGGPRICLGQQFALTEVGYVLSR
LVQTYETLETCDHKPLPPLYNHALTMCHEEGVWVKMYKGEKA
ALK6:
(SEQ ID NO: 6)
MIQSVFLALAILIAYLGFAEWFSRFQHRRISKKKGCGMPPMANGGFLGWYGLYK
TYQITSERTYPHSMRMGLEAFGHTFVYPVPGTDMLQTIHPDNIKAILATQFKDFS
LGTRHKIMLPTLGDGIFTLDGEGWTHSRALLRPQFARDQVSHVASLERHIQVLFK
TIKKENKECDPAKGFDIQELFFMLTLDTATEFLCGDSVDSLTDYLADPTAPQLDH
SGIDENVRRAFPEAFNTAQWFCSIRAKLMKLYFFAGTVFYRKKYADANKIVHDF
TDFYVSKALAARKEKFQELDQEGKYIFLYELAKETRNPKVLRDQMLNILLAGRD
TTASLLSWVMFRMARQPETWKKLRQAVINDFGDTPDELSFESLKRCEYLRYVLN
EGLRLYPSVPMNFRVATRDTTLPKGGGPDLDQPIFIPKGGIVVYSVYHTHRAEEY
WGKDTEEFIPERWDPAEGYQIARGWEYLPFNGGPRICLGQQFALTEAGYVLARL
AQEFETVTSCDDKPLPPKYNTHLTMSHDDGVWLKME
ALK7:
(SEQ ID NO: 7)
MFQLFSILVLAFTTALVAQLAYNQYDYQRKVKKFGCGQLRVAENGLFGWKGLR
EVLRINKYKLGPAALKDRFEKYGKTHVFHVGPSPLITTMDPENIKAMLATQFKDF
CLIARYKALGPMLGDGIFTLDGHGWTHSRALLRPQFAREQVSRLDSIEHHFQILK
KCISKEMSDKRDTQRGFDIQNLFFLMTLDTATEFLFGSSVDSLVDFLDDPSIQTGD
HGGIDEAARKGFSNAFNRAQELSSLRTRLHKLYWVIGTLAVREPYHRYNREVKT
FVDHYAAKAIKARNEKNTDLLDNDKYIFMYELVKETSNPITLRDQMLNILLAGR
DTTASMLSWIYFRLARDPKRYAKLRAAVLADFGPGPENITFESLKKCDYLRYVL
NESLRVYPVVPINARTASRDTTLPRGGGPDGSQPIFVPKGQTVSYSVWWTHRDPE
FWGQDAEEFIPERWDTKNGSIGRGWEYLPFNGGPRICLGQQFALTEVGYVLSRM
VQTYETLESGDTKPLPPLYNHALTLCHQEGVWIKTE
CP124:
(SEQ ID NO: 8)
MGLNTAIATRVNGTPPPEVPIADIELGSLDFWALDDDVRDGAFATLRREAPISFW
PTIELPGFVAGNGHWALTKYDDVFYASRHPDIFSSYPNITINDQTPELAEYFGSMI
VLDDPRHQRLRSIVSRAFTPKVVARIEAAVRDRAHRLVSSMIANNPDRQADLVSE
LAGPLPLQIICDMMGIPKADHQRIFHWTNVILGFGDPDLATDFDEFMQVSADIGA
YATALAEDRRVNHHDDLTSSLVEAEVDGERLSSREIASFFILLVVAGNETTRNAIT
HGVLALSRYPEQRDRWWSDFDGLAPTAVEEIVRWASPVVYMRRTLTQDIELRG
TKMAAGDKVSLWYCSANRDESKFADPWTFDLARNPNPHLGFGGGGAHFCLGA
NLARREIRVAFDELRRQMPDVVATEEPARLLSQFIHGIKTLPVTWS
CP2E1:
(SEQ ID NO: 9)
MSALGVTVALLVWAAFLLLVSMWRQVHSSWNLPPGPFPLPIIGNLFQLELKNIPK
SFTRLAQRFGPVFTLYVGSQRMVVMHGYKAVKEALLDYKDEFSGRGDLPAFHA
HRDRGIIFNNGPTWKDIRRFSLTTLRNYGMGKQGNESRIQREAHFLLEALRKTQG
QPFDPTFLIGCAPCNVIADILFRKHFDYNDEKFLRLMYLENENFHLLSTPWLQLY
NNFPSFLHYLPGSHRKVIKNVAEVKEYVSERVKEHHQSLDPNCPRDLTDCLLVE
MEKEKHSAERLYTMDGITVTVADLFFAGTETTSTTLRYGLLILMKYPEIEEKLHE
EIDRVIGPSRIPAIKDRQEMPYMDAVVHEIQRFITLVPSNLPHEATRDTIFRGYLIP
KGTVVVPTLDSVLYDNQEFPDPEKFKPEHFLNENGKFKYSDYFKPFSTGKRVCA
GEGLARMELFLLLCAILQHFNLKPLVDPKDIDLSPIHIGFGCIPPRYKLCVIPRS
AOX:
(SEQ ID NO: 10)
MSDDKHTFDFIIVGGGTAGPTLARRLADAWISGKKLKVLLLESGPSSEGVDDIRC
PGNWVNTIHSEYDWSYEVDEPYLSTDGEERRLCGIPRGHCLGGSSCLNTSFVIRG
TRGDFDRIEEETGAKGWGWDDLFPYFRKHECYVPQGSAHEPKLIDFDTYDYKKF
HGDSGPIKVQPYDYAPISKKFSESLASFGYPYNPEIFVNGGAPQGWGHVVRSTSN
GVRSTGYDALVHAPKNLDIVTGHAVTKILFEKIGGKQTAVGVETYNRAAEEAGP
TYKARYEVVVCCGSYASPQLLMVSGVGPKKELEEVGVKDIILDSPYVGKNLQDH
LICGIFVEIKEPGYTRDHQFFDDEGLDKSTEEWKTKRTGFFSNPPQGIFSYGRIDNL
LKDDPVWKEACEKQKALNPRRDPMGNDPSQPHFEIWNAELYIELEMTQAPDEG
QSVMTVIGEILPPRSKGYVKLLSPDPMENPEIVHNYLQDPVDARVFAAIMKHAA
DVATNGAGTKDLVKARWPPESKPFEEMSIEEWETYVRDKSHTCFHPCGTVKLG
GANDKEAVVDERLRVKGVDGLRVADVSVLPRVPNGHTQAFAYAVGEKAADLIL
ADIAGKDLRPRI
AP:
(SEQ ID NO: 11)
MDQKTDSREYLDGFKAGQKVAYERLYWRRILVLFGGLAMVLGFFGYKGYAVN
DVLDLDLSMSRADSVNDNPLVSPLSKPKKNVIFMVTDGMGPASVSLARTYRQY
VQGLAYNNTLTIDKHFIGSSRTRSSDSPVTDSAAGATAFSCGAKSYNGAISVTPD
YKACGSVLEAAKRQGLKTGLVVTTRITDATPACFGAHVAHRSQEDEIADQLLGY
ATPLGRSVDLLMGGGRIHFTTKGRQDGRDLIAEAQIDGFQYIANRKEFDELDTSN
ATLPLLALFTDYDMPYEIDRVPEQFPSLKETAISALEILHQETKDSDEGFFIMIEGS
RIDHAGHQNDPAAQVREVMAFDEMFAAVVDFADSLDTETIIVSTSDHETGGLAI
ARQVQAEYPEYVWYPEALAAAQHSGEYIARKLQAFARPDHPSEASGSKLEKFV
KREILENDLGVKDYTKQEVKALIRNRADPIDTIVDIVSRRSQTGWSTHGHSAVDV
NIYAHSNKQSGLDKLDALRGNHENIEIGQFLAKYLEVDVARVTEILEDLPVRVQH
VEEIDDCDQYHHGLY
DP1:
(SEQ ID NO: 12)
MTYSTQSSSTLVAIRRDECFFPISSHSISLSSVLSYLIDWIFYISLTTLALVYAKIVSP
LFAEFYLYNTSLWYSHIPTDLTIVPTFLLIIYSILIPIGQFALTIGFTTSHRWHRRLW
DLHAILLTLMAAHALQTVIVSLLKNLVGAPRPDMLARCRPMSWMRPSFGTLSNV
GICTQTDIGHLEEGFRSFPSAHSATAFTSAMVQVLFWIARTRMLDCSGWSWKLL
LSLVPLLSASAVAFSRISDNRHHVFDVIIGMLIGLIAGYLAFIHYFPFPTFANVCTG
GRAYSPRCGILGSVGCWSLGDETGCLRTKLFKTPKCSVKATFTMGCGTELCVCK
KPSCGACSVGGEGAKCSLRCSIRNCTGNACTSRPVSRRSTRRTPSRRSRRSHQTY
PGRSSETACLPSCSPTCTGNCHDTTCSSCDSSATATEPSHTRSAHRCTNPVCTIAG
CIGACLRRVTSRPRCTTVGCTVANCIGISCLVGRVRTCRIPWCRNERCMRLAREH
CFDEEASIIDVGGGRSRASRHRRVSRHAITP
DP2:
(SEQ ID NO: 13)
MLSSSSTLKQYHVIEWITAFTLIFLWYVSESAAPFTREFIISDPTINHSHVTVERVSS
EACILYTIIIPFFVLIGLSAIMAPRPQDRLKFMSITLSTFLVAAFFNGFITNFLKIYMG
RHRPDFIARCEPSKRAPIDKYVTIEVCTGDMDTILEGMKSTPSGHSSTAFVGMTFF
CLWVYGQINAYKTYGSKASKLLLAFFPLLLAIYIALSRTEDYRHHFVDIVLGSLL
GMTIAYYFYRREFPRTTSKTSHIPYCLGSEGADDDHHYDRVDDVQLENQV

### REFERENCES

1. Grabińska, K. & Palamarczyk, G. Dolichol biosynthesis in the yeast Saccharomyces cerevisiae: an insight into the regulatory role of farnesyl diphosphate synthase. FEMS Yeast Res. 2, 259-265 (2002).
2. Iida, T., Sumita, T., Ohta, A. & Takagi, M. The cytochrome P450ALK multigene family of an n-alkane-assimilating yeast, Yarrowia lipolytica: cloning and characterization of genes coding for new CYP52 family members. Yeast 16, 1077-1087 (2000).
3. Fickers, P. et al. Hydrophobic substrate utilisation by the yeast Yarrowia lipolytica, and its potential applications. FEMS Yeast Res. 5, 527-543 (2005).
4. Gatter, M. et al. A newly identified fatty alcohol oxidase gene is mainly responsible for the oxidation of long-chain ω-hydroxy fatty acids in Yarrowia lipolytica. FEMS Yeast Res. 14, 858-872 (2014).
5. Johnston, J. B., Kells, P. M., Podust, L. M. & Ortiz de Montellano, P. R. Biochemical and structural characterization of CYP124: A methyl-branched lipid ω-hydroxylase from Mycobacterium tuberculosis. Proc. Natl. Acad. Sci. 106, 20687-20692 (2009).
6. FUKUDA, R. Metabolism of Hydrophobic Carbon Sources and Regulation of It in n-Alkane-Assimilating Yeast Yarrowia lipolytica. Biosci. Biotechnol. Biochem. 77, 1149-1154 (2013).
7. Iwama, R. et al. Functional roles and substrate specificities of twelve cytochromes P450 belonging to CYP52 family in n-alkane assimilating yeast Yarrowia lipolytica. Fungal Genet. Biol. FG B 91, 43-54 (2016).
8. Schwartz, C., Shabbir-Hussain, M., Frogue, K., Blenner, M. & Wheeldon, I. Standardized Markerless Gene Integration for Pathway Engineering in Yarrowia lipolytica. ACS Synth. Biol. 6, 402-409 (2017).
9. Seitzer, P., Bennett, B. & Melamud, E. MAVEN2: An Updated Open-Source Mass Spectrometry Exploration Platform. Metabolites 12, 684 (2022).
10. Gennaiou, K., Kelesidis, A. & Zografos, A. L. Climbing the Oxidase Phase Ladder by Using Dioxygen as the Sole Oxidant: The Case Study of Costunolide. Org. Lett. 26, 2934-2938 (2024).

### CONCLUSION

This concludes the description of the illustrative embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A composition of matter comprising a bifunctional terpenoid and a microorganism engineered to overexpress β-hydroxy β-methylglutaryl-CoA reductase and farnesyl pyrophosphate synthase.

2. The composition of claim 1, wherein the microorganism is further engineered to overexpress ALK3, ALK4, ALK5, ALK6 and/or ALK7 and oxidases and dehydrogenases, such as AOX, ALDH2, ADH1 and/or ADH3.

3. The composition of claim 1, wherein the bifunctional terpenoid comprises an isoprenoic diacid, a geranoic diacid, a farnesoid diacid, a geranylgeranoic diacid and/or a geranylfarnesoic diacid as well as their dihydro versions.

4. The composition of claim 1, wherein the bifunctional terpenoid comprises at least one compound comprising a structure: 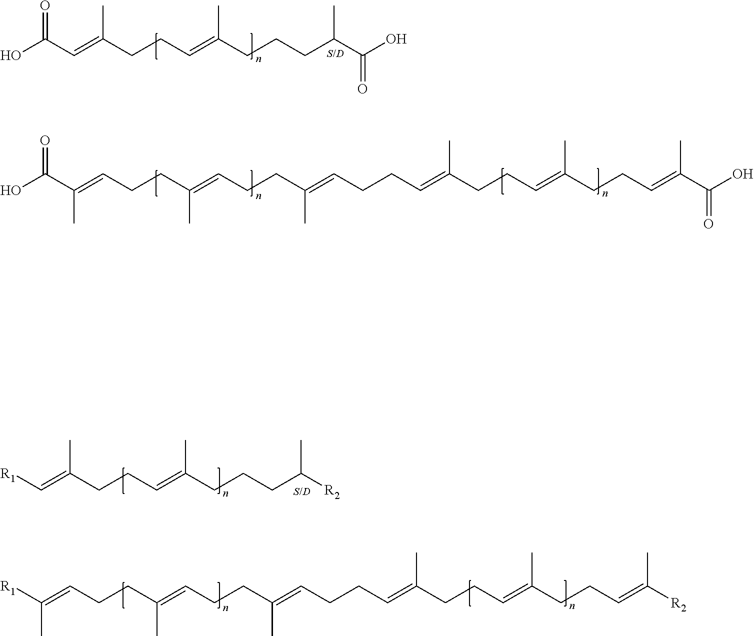
where R₁ and R₂ are each CH₃, CH₂OH, CHO, or CO₂H, and n is a whole number.

5. A composition of matter comprising microorganisms making a bifunctional terpenoid, wherein when combined with a culture media at 30° C., the microorganism makes the bifunctional terpenoid such that concentrations of the bifunctional terpenoid is at least 0.1, 0.5, 1 or 10 milligrams/L in the microorganisms within the culture media.

6. The composition of claim 5, wherein the microorganism is a yeast.

7. The composition of claim 5, wherein the microorganism is a Yarrowia, Saccharomyces, Candida, Rhodosporidium, Cryptococcus, Rhodotorula, Lipomyces, or Trichosporon yeast species or an Escherichia, Methylobacterium, or Rhodococcus bacteria species.

8. The composition of claim 5, wherein the microorganism comprises exogenous/altered nucleic acid sequences that increase the expression of β-hydroxy β-methylglutaryl-CoA reductase and farnesyl pyrophosphate synthase in the microorganism.

9. The composition of claim 8, wherein the microorganism comprises exogenous/altered nucleic acid sequences that increase the expression of ALK3, ALK4, ALK5, ALK6 and/or ALK7 and AOX, ALDH2, ADH1 and/or ADH3 polypeptides in the microorganism.

10. The composition of claim 5, wherein:
the culture media does not include glucose as a carbon source;
the culture media includes gluconate and/or acetate; and/or
the culture media includes one or more agents selected to increase the production of bifunctional terpenoids.

11. The composition of claim 5, wherein the bifunctional terpenoid comprises a compound having a formula: 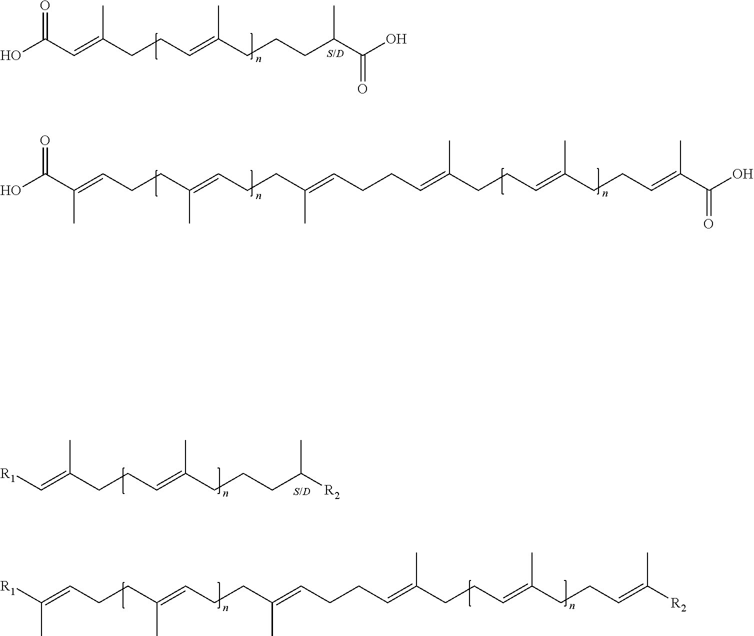
where R₁ and R₂ are each CH₃, CH₂OH, CHO, or CO₂H, and n is a whole number.

12. A method of making a bifunctional terpenoid comprising:
combining a microorganism with a culture media, wherein:
the microorganism is selected to comprise exogenous/altered nucleic acid sequences selected to increase the expression of β-hydroxy β-methylglutaryl-CoA reductase and farnesyl pyrophosphate synthase polypeptides in the microorganism; and
the culture media is selected to allow the production of the bifunctional terpenoid when the microorganism is disposed therein;
such that the bifunctional terpenoid is made.

13. The method of claim 12, wherein the microorganism is further selected to comprise exogenous/altered nucleic acid sequences selected to increase the expression of ALK3, ALK4, ALK5, ALK6, ALK7, AOX, ALDH2, ADH1 and/or ADH3 polypeptides in the engineered microorganism.

14. The method of claim 12, wherein the microorganism is a Yarrowia, Saccharomyces, Candida, Rhodosporidium, Cryptococcus, Rhodotorula, Lipomyces, or Trichosporon yeast species or an Escherichia, Methylobacterium, or Rhodococcus bacteria species.

15. The method of claim 12, wherein the culture media comprises a YPD culture media, a YNB culture media, LB culture media, or M9 culture media.

16. The method of claim 12, wherein amounts of the bifunctional terpenoid within microorganism cells in the culture media are at least 0.1, 0.5, 1 or 10 milligrams/L.

17. The method of claim 12, further comprising performing a purification process on the bifunctional terpenoid.

18. The method of claim 17, further comprising performing a polymerization process on the bifunctional terpenoid.

19. The method of claim 17, further comprising performing a functionalization and/or cyclization process on the bifunctional terpenoid.

20. The method of claim 12, wherein:
the culture media does not include glucose as a carbon source;
the culture media includes gluconate and/or acetate; and/or
the culture media includes one or more agents selected to increase the production of bifunctional terpenoids.

21. A bifunctional terpenoid made by the method of claim 12.

22. A composition of matter comprising at least one bifunctional terpenoid compound shown in FIG. 8.

23. The composition of claim 22, further comprising a microorganism engineered to overexpress β-hydroxy β-methylglutaryl-CoA reductase and farnesyl pyrophosphate synthase.


SEQUENCE LISTING (13 sequences)

SEQ ID NO: 1 - protein, 999 aa, unidentified
  source 1..999: note = Yarrowia lipolytica
MLQAAIGKIVGFAVNRPIHTVVLTSIVASTAYLAILDIAIPGFEGTQPISYYHPAAKSYDNPADWTHIAEADIPSDAYRLAFAQIRVSDVQGGEAPTIPGAVAVSDLDHRIVMDYKQWAPWTASNEQIASENHIWKHSFKDHVAFSWIKWFRWAYLRLSTLIQGADNFDIAVVALGYLAMHYTFFSLFRSMRKVGSHFWLASMALVSSTFAFLLAVVASSSLGYRPSMITMSEGLPFLVVAIGFDRKVNLASEVLTSKSSQLAPMVQVITKIASKALFEYSLEVAALFAGAYTGVPRLSQFCFLSAWILIFDYMFLLTFYSAVLAIKFEINHIKRNRMIQDALKEDGVSAAVAEKVADSSPDAKLDRKSDVSLFGASGAIAVFKIFMVLGFLGLNLINLTAIPHLGKAAAAAQSVTPITLSPELLHAIPASVPVVVTFVPSVVYEHSQLILQLEDALTTFLAACSKTIGDPVISKYIFLCLMVSTALNVYLFGATREVVRTQSVKVVEKHVPIVIEKPSEKEEDTSSEDSIELTVGKQPKPVTETRSLDDLEAIMKAGKTKLLEDHEVVKLSLEGKLPLYALEKQLGDNTRAVGIRRSIISQQSNTKTLETSKLPYLHYDYDRVFGACCENVIGYMPLPVGVAGPMNIDGKNYHIPMATTEGCLVASTMRGCKAINAGGGVTTVLTQDGMTRGPCVSFPSLKRAGAAKIWLDSEEGLKSMRKAFNSTSRFARLQSLHSTLAGNLLFIRFRTTTGDAMGMNMISKGVEHSLAVMVKEYGFPDMDIVSVSGNYCTDKKPAAINWIEGRGKSVVAEATIPAHIVKSVLKSEVDALVELNISKNLIGSAMAGSVGGFNAHAANLVTAIYLATGQDPAQNVESSNCITLMSNVDGNLLISVSMPSIEVGTIGGGTILEPQGAMLEMLGVRGPHIETPGANAQQLARIIASGVLAAELSLCSALAAGHLVQSHMTHNRSQAPTPAKQSQADLQRLQNGSNICIRS

SEQ ID NO: 2 - protein, 344 aa, unidentified
  source 1..344: note = Yarrowia lipolytica
MSKAKFESVFPRISEELVQLLRDEGLPQDAVQWFSDSLQYNCVGGKLNRGLSVVDTYQLLTGKKELDDEEYYRLALLGWLIELLQAFFLVSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 3 - protein, 513 aa, unidentified
  source 1..513: note = Yarrowia lipolytica
MIIIETLIGAVVFVAVYVAFVKLDYYRRKAKFETSDMPVAYNGLLGWKGLRHMLTVFNNDIGPVGWREVFATYGKTLKYYAFPSNTILTYDPDNIKAMLATQFKDFSLGLRKEALAPSLGYGIFTLDGSSWSHSRALLRPQFSREQISRLESVETHVQEMMSCIDRNQGAYFDIQRLFFSLAMDTATDFLLGEAVGNLQEILHPEMPRTGTTFQVAFDRAQRLGSLRIICQEAFWVVGSLFWRRDFNNTNQHIHDYVDRYVDKALLARKEKSEIYTNPDKYIFLYELARETTNKITLRDQVLNILIAGRDTTASTLSWIFMELAKKPDIFHKLREAILNDFGTSCESISFESLKKCDYLRQVLNEGLRLHPVVPVNLRVAVRDTTLPRGGGPQGDKPIFVAKGQKINYAIFWTHRDKEYWGEDAEEFRPERWETTSGGALGKGWEFLPFNGGPRICLGQQFALTEMGYVITRLLQEYSDISIQPSDAAVKVRHSLTMCSAQGINISLTRAKEE

SEQ ID NO: 4 - protein, 519 aa, unidentified
  source 1..519: note = Yarrowia lipolytica
MLTNLTIVLITLLVTYTVLTRTALRIQRARKAKQMGATLPPRVNNGILGWYGLWLVIQNARSMKLPHTLGKRFANGPTWLTPVAGNEPINTIDPENVKAILATQFKDFCLGIRHRALSPSIGDGIFTLDGEGWTHSRALLRPQFSRQQISRVHSLERLMQILFKLIRKENGEYFDLQNLFFMFTLDSATEFLYGASVDTLADLLGEPVEGDHGGVGEEVRKAYQQSINNAQDISAIRTRLQGLYWIAGNIYQRNLYQKSNKGVKDFSQFFVDKALNTSKEKLKEMEDSDNYVFLYELVKSTRNPVVIRDQLINILVAGRDTTASLLSFTFYTLGRRPDVLKKLRAAILEDFGTSPDEITFESLKRCDYLRYVLNEVLRLYPSVPINARSATRDTTLPRGGGPDGKQPVFVYKGQMVAYCVYWMHRDKKYWGEDALEFNPDRWDPKVQPQNKGWEYLPFNGGPRICLGQQFALTEAGYVVTRMLQEFDTVHCKNQKEEEHPPYALDLTMRHGEGVWVSMK

SEQ ID NO: 5 - protein, 531 aa, unidentified
  source 1..531: note = Yarrowia lipolytica
MLQLFGVLVLALTTALLAQLAYNKYEYNRKVKQFGCGELTVAKNGFLGWKGIRAVLHVLKTKKGPAALKERIDAYGRTYVFHIGPAPVISTMEPENIKAMLATQFKDFSLGTRYRSLAPTLGDGIFTLDGHGWTHSRALLRPQFAREQVSRLDSLEAHFQILKMCVDKEMREKGNDPRGFDIQNLFFLFTLDSATEFLFGSSVDSLVDFLDDPSVRTGDHGGVDEAARKGFNNSFNHAQELCALRSRLHTLYWIVGSVVKKEPFERYNKEIKTFVDFFAAKALKARKEKDMSLMDNDQYIFMYELVKETTNPVTLRDQMLNILLAGRDTTASMLSWIYFRLARDPKLYAKLRSAILEDFGTTPEAITFESLKQCDYLRYVLNEALRLYPVVPINGRTATRDTTLPRGGGPDQSQPIFIPKGQTVSYSVYWTHRDPRFWGEDAEEFIPERWDPRNGNIGRGWEYLPFNGGPRICLGQQFALTEVGYVLSRLVQTYETLETCDHKPLPPLYNHALTMCHEEGVWVKMYKGEKA

SEQ ID NO: 6 - protein, 526 aa, unidentified
  source 1..526: note = Yarrowia lipolytica
MIQSVFLALAILIAYLGFAEWFSRFQHRRISKKKGCGMPPMANGGFLGWYGLYKTYQITSERTYPHSMRMGLEAFGHTFVYPVPGTDMLQTIHPDNIKAILATQFKDFSLGTRHKIMLPTLGDGIFTLDGEGWTHSRALLRPQFARDQVSHVASLERHIQVLFKTIKKENKECDPAKGFDIQELFFMLTLDTATEFLCGDSVDSLTDYLADPTAPQLDHSGIDENVRRAFPEAFNTAQWFCSIRAKLMKLYFFAGTVFYRKKYADANKIVHDFTDFYVSKALAARKEKFQELDQEGKYIFLYELAKETRNPKVLRDQMLNILLAGRDTTASLLSWVMFRMARQPETWKKLRQAVINDFGDTPDELSFESLKRCEYLRYVLNEGLRLYPSVPMNFRVATRDTTLPKGGGPDLDQPIFIPKGGIVVYSVYHTHRAEEYWGKDTEEFIPERWDPAEGYQIARGWEYLPFNGGPRICLGQQFALTEAGYVLARLAQEFETVTSCDDKPLPPKYNTHLTMSHDDGVWLKME

SEQ ID NO: 7 - protein, 526 aa, unidentified
  source 1..526: note = Yarrowia lipolytica
MFQLFSILVLAFTTALVAQLAYNQYDYQRKVKKFGCGQLRVAENGLFGWKGLREVLRINKYKLGPAALKDRFEKYGKTHVFHVGPSPLITTMDPENIKAMLATQFKDFCLIARYKALGPMLGDGIFTLDGHGWTHSRALLRPQFAREQVSRLDSIEHHFQILKKCISKEMSDKRDTQRGFDIQNLFFLMTLDTATEFLFGSSVDSLVDFLDDPSIQTGDHGGIDEAARKGFSNAFNRAQELSSLRTRLHKLYWVIGTLAVREPYHRYNREVKTFVDHYAAKAIKARNEKNTDLLDNDKYIFMYELVKETSNPITLRDQMLNILLAGRDTTASMLSWIYFRLARDPKRYAKLRAAVLADFGPGPENITFESLKKCDYLRYVLNESLRVYPVVPINARTASRDTTLPRGGGPDGSQPIFVPKGQTVSYSVWWTHRDPEFWGQDAEEFIPERWDTKNGSIGRGWEYLPFNGGPRICLGQQFALTEVGYVLSRMVQTYETLESGDTKPLPPLYNHALTLCHQEGVWIKTE

SEQ ID NO: 8 - protein, 428 aa, unidentified
  source 1..428: note = Yarrowia lipolytica
MGLNTAIATRVNGTPPPEVPIADIELGSLDFWALDDDVRDGAFATLRREAPISFWPTIELPGFVAGNGHWALTKYDDVFYASRHPDIFSSYPNITINDQTPELAEYFGSMIVLDDPRHQRLRSIVSRAFTPKVVARIEAAVRDRAHRLVSSMIANNPDRQADLVSELAGPLPLQIICDMMGIPKADHQRIFHWTNVILGFGDPDLATDFDEFMQVSADIGAYATALAEDRRVNHHDDLTSSLVEAEVDGERLSSREIASFFILLVVAGNETTRNAITHGVLALSRYPEQRDRWWSDFDGLAPTAVEEIVRWASPVVYMRRTLTQDIELRGTKMAAGDKVSLWYCSANRDESKFADPWTFDLARNPNPHLGFGGGGAHFCLGANLARREIRVAFDELRRQMPDVVATEEPARLLSQFIHGIKTLPVTWS

SEQ ID NO: 9 - protein, 493 aa, unidentified
  source 1..493: note = Yarrowia lipolytica
MSALGVTVALLVWAAFLLLVSMWRQVHSSWNLPPGPFPLPIIGNLFQLELKNIPKSFTRLAQRFGPVFTLYVGSQRMVVMHGYKAVKEALLDYKDEFSGRGDLPAFHAHRDRGIIFNNGPTWKDIRRFSLTTLRNYGMGKQGNESRIQREAHFLLEALRKTQGQPFDPTFLIGCAPCNVIADILFRKHFDYNDEKFLRLMYLFNENFHLLSTPWLQLYNNFPSFLHYLPGSHRKVIKNVAEVKEYVSERVKEHHQSLDPNCPRDLTDCLLVEMEKEKHSAERLYTMDGITVTVADLFFAGTETTSTTLRYGLLILMKYPEIEEKLHEEIDRVIGPSRIPAIKDRQEMPYMDAVVHEIQRFITLVPSNLPHEATRDTIFRGYLIPKGTVVVPTLDSVLYDNQEFPDPEKFKPEHFLNENGKFKYSDYFKPFSTGKRVCAGEGLARMELFLLLCAILQHFNLKPLVDPKDIDLSPIHIGFGCIPPRYKLCVIPRS

SEQ ID NO: 10 - protein, 609 aa, unidentified
  source 1..609: note = Yarrowia lipolytica
MSDDKHTFDFIIVGGGTAGPTLARRLADAWISGKKLKVLLLESGPSSEGVDDIRCPGNWVNTIHSEYDWSYEVDEPYLSTDGEERRLCGIPRGHCLGGSSCLNTSFVIRGTRGDFDRIEEETGAKGWGWDDLFPYFRKHECYVPQGSAHEPKLIDFDTYDYKKFHGDSGPIKVQPYDYAPISKKFSESLASFGYPYNPEIFVNGGAPQGWGHVVRSTSNGVRSTGYDALVHAPKNLDIVTGHAVTKILFEKIGGKQTAVGVETYNRAAEEAGPTYKARYEVVVCCGSYASPQLLMVSGVGPKKELEEVGVKDIILDSPYVGKNLQDHLICGIFVEIKEPGYTRDHQFFDDEGLDKSTEEWKTKRTGFFSNPPQGIFSYGRIDNLLKDDPVWKEACEKQKALNPRRDPMGNDPSQPHFEIWNAELYIELEMTQAPDEGQSVMTVIGEILPPRSKGYVKLLSPDPMENPEIVHNYLQDPVDARVFAAIMKHAADVATNGAGTKDLVKARWPPESKPFEEMSIEEWETYVRDKSHTCFHPCGTVKLGGANDKEAVVDERLRVKGVDGLRVADVSVLPRVPNGHTQAFAYAVGEKAADLILADIAGKDLRPRI

SEQ ID NO: 11 - protein, 558 aa, unidentified
  source 1..558: note = Yarrowia lipolytica
MDQKTDSREYLDGFKAGQKVAYERLYWRRILVLFGGLAMVLGFFGYKGYAVNDVLDLDLSMSRADSVNDNPLVSPLSKPKKNVIFMVTDGMGPASVSLARTYRQYVQGLAYNNTLTIDKHFIGSSRTRSSDSPVTDSAAGATAFSCGAKSYNGAISVTPDYKACGSVLEAAKRQGLKTGLVVTTRITDATPACFGAHVAHRSQEDEIADQLLGYATPLGRSVDLLMGGGRIHFTTKGRQDGRDLIAEAQIDGFQYIANRKEFDELDTSNATLPLLALFTDYDMPYEIDRVPEQFPSLKETAISALEILHQETKDSDEGFFIMIEGSRIDHAGHQNDPAAQVREVMAFDEMFAAVVDFADSLDTETIIVSTSDHETGGLAIARQVQAEYPEYVWYPEALAAAQHSGEYIARKLQAFARPDHPSEASGSKLEKFVKREILENDLGVKDYTKQEVKALIRNRADPIDTIVDIVSRRSQTGWSTHGHSAVDVNIYAHSNKQSGLDKLDALRGNHENIEIGQFLAKYLEVDVARVTEILEDLPVRVQHVEEIDDCDQYHHGLY

SEQ ID NO: 12 - protein, 533 aa, unidentified
  source 1..533: note = Yarrowia lipolytica
MTYSTQSSSTLVAIRRDECFFPISSHSISLSSVLSYLIDWIFYISLTTLALVYAKIVSPLFAEFYLYNTSLWYSHIPTDLTIVPTFLLIIYSILIPIGQFALTIGFTTSHRWHRRLWDLHAILLTLMAAHALQTVIVSLLKNLVGAPRPDMLARCRPMSWMRPSFGTLSNVGICTQTDIGHLEEGFRSFPSAHSATAFTSAMVQVLFWIARTRMLDCSGWSWKLLLSLVPLLSASAVAFSRISDNRHHVFDVIIGMLIGLIAGYLAFIHYFPFPTFANVCTGGRAYSPRCGILGSVGCWSLGDETGCLRTKLFKTPKCSVKATFTMGCGTELCVCKKPSCGACSVGGEGAKCSLRCSIRNCTGNACTSRPVSRRSTRRTPSRRSRRSHQTYPGRSSETACLPSCSPTCTGNCHDTTCSSCDSSATATEPSHTRSAHRCTNPVCTIAGCIGACLRRVTSRPRCTTVGCTVANCIGISCLVGRVRTCRIPWCRNERCMRLAREHCFDEEASIIDVGGGRSRASRHRRVSRHAITP

SEQ ID NO: 13 - protein, 278 aa, unidentified
  source 1..278: note = Yarrowia lipolytica
MLSSSSTLKQYHVIEWITAFTLIFLWYVSESAAPFTREFIISDPTINHSHVTVERVSSEACILYTIIIPFFVLIGLSAIMAPRPQDRLKFMSITLSTFLVAAFFNGFITNFLKIYMGRHRPDFIARCEPSKRAPIDKYVTIEVCTGDMDTILEGMKSTPSGHSSTAFVGMTFFCLWVYGQINAYKTYGSKASKLLLAFFPLLLAIYIALSRTEDYRHHFVDIVLGSLLGMTIAYYFYRREFPRTTSKTSHIPYCLGSEGADDDHHYDRVDDVQLENQV